(19) United States
(12) Patent Application Publication
(10) Pub. No.: US 2008/0241086 A1
(43) Pub. Date: Oct. 2, 2008

(54) Line of cosmetic compositions

(75) Inventor: Ludovic Thevenet, Bourg La Reine (FR)

Correspondence Address: OLIFF & BERRIDGE, PLC, P.O. BOX 320850, ALEXANDRIA, VA, 22320-4850, US

(73) Assignee: L'OREAL, PARIS (FR)

(21) Appl. No.: 11/984,193

(22) Filed: Nov. 14, 2007

Related U.S. Application Data
(60) Provisional application No. 60/875,555, filed on Dec. 19, 2006.
(60) Provisional application No. 60/875,554, filed on Dec. 19, 2006.
(60) Provisional application No. 60/875,549, filed on Dec. 19, 2006.
(60) Provisional application No. 60/876,160, filed on Dec. 21, 2006.
(60) Provisional application No. 60/876,161, filed on Dec. 21, 2006.
(60) Provisional application No. 60/876,162, filed on Dec. 21, 2006.

(30) Foreign Application Priority Data
Nov. 17, 2006 (FR) 06 54975
Nov. 17, 2006 (FR) 06 54977
Nov. 17, 2006 (FR) 06 54979
Nov. 17, 2006 (FR) 06 54980
Nov. 17, 2006 (FR) 06 54981
Nov. 17, 2006 (FR) 06 54982
Dec. 12, 2006 (FR) 06 55452
Dec. 12, 2006 (FR) 06 55454
Dec. 12, 2006 (FR) 06 55457
Dec. 12, 2006 (FR) 06 55458
Dec. 12, 2006 (FR) 06 55459
Dec. 12, 2006 (FR) 06 55460

(51) Int. Cl. A61K 8/18; A61Q 19/00

(52) U.S. Cl. 424/63

(57) ABSTRACT

The present invention relates to a cosmetic composition including, in a cosmetically-acceptable medium, at least one multilayer interference pigment and at least one coloring agent that is sensitive to at least one external stimulus, the coverage of the composition being greater than or equal to 25, better 30, the multilayer interference pigment being at a content that imparts to the composition, a difference in color ΔE between the bulk color and the color after application that is greater than or equal to 5.

This non provisional application claims the benefit of French Applications No^{s}. 06 54982-06 54979-06 54980-06 54981-06 54977 and 06 54975 filed on Nov. 17, 2006 and of French Applications No^{s}. 06 55459-06 55457-06 55458-06 55460-06 55454 and 06 55452 filed on Dec. 12, 2006 and U.S. Provisional Applications Nos. 60/875,555-60/875,554 and 60/875,549 filed on Dec. 19, 2006 and U.S. Provisional Applications Nos. 60/876,160-60/876,161 and 60/876,162 filed on Dec. 21, 2006.

The present invention relates to cosmetic compositions, and more particularly to compositions for making up the skin, the lips, hair, or the nails.

### BACKGROUND

It is known for makeup compositions to incorporate coloring agents having a color that is sensitive to an external stimulus, the coloring agents also being referred to as “Xchrome” coloring agents, with X designating the external factor. By way of example, the coloring agents can be photochromic, thermochromic, solvatochromic, or tribochromic coloring agents.

It is known for makeup compositions to incorporate diffracting pigments in order to produce an iridescent effect or an effect of diffracting the light that produces a color variation depending on the angle of incidence of the light or of observation.

It is known to introduce diffusing pigments into makeup compositions so as to produce colors by a phenomenon of absorbing light using specific chromophores.

It is known to introduce diffusing pigments into makeup compositions so as to produce colors by a phenomenon of absorbing light using specific chromophores.

To the diffusing pigments that are necessary in order to benefit from a colored background that is continuous and sufficiently covering, it is possible to add effect particles in order to create highlights or to impart a nacre aspect, for example.

However, the intensity of the color produced by such compositions can turn out to be insufficient for obtaining a completely satisfactory result.

Multilayer interference pigments, comprising a stack of layers that have refractive indices and thicknesses that are conveniently selected in order to generate a color by an interference phenomenon, make it possible to produce a color of intensity that is greater than the color intensity of the above-mentioned diffusing pigments.

To the inventor's knowledge, in commercially-available compositions, and with the exception of powders, such multilayer interference pigments are used at a concentration by weight that does not exceed 5%.

In addition, it is known from eyeshadows of trade name CHRYSALIDE by LANCÔME to impart both coverage and color intensity by means of makeup that is applied in a first operation of depositing, on the keratinous materials, a black base coat containing a diffusing pigment that provides coverage, and then, in a second operation of depositing, on the base coat, a composition that provides color by means of a multilayer interference pigment. Without the base coat, the top coat is practically invisible since it has no covering powder and no color.

The need for two successive applications complicates applying the makeup and makes packaging it more costly.

### SUMMARY

Composition with a coloring agent that is sensitive to at least one external stimulus or with a reflecting pigment with metallic reflection or with a diffracting pigment.

A need exists to benefit from a composition that is capable of providing coverage and of producing a saturated color, so as to make it possible, in a single operation, to obtain makeup that is both covering and colored.

There exists a need to have a composition that presents novel effects.

In addition, it is desirable to benefit from making up keratinous materials with cosmetic compositions that present novel effects that are likely to attract consumers, without the novel effects being to the detriment of the quality of the makeup obtained.

The invention seeks in particular to satisfy all or some of the above-identified needs.

In one of its aspects, the invention provides a cosmetic composition including, in a cosmetically-acceptable medium, at least one multilayer interference pigment and at least one of:
a coloring agent that is sensitive to at least one external stimulus, or
a reflecting pigment with metallic reflection, or
a diffracting pigment,
   the coverage of the composition being greater than or equal to 25, better 30, the multilayer interference pigment being at a content that imparts to the composition, a difference in color ΔE between the bulk color and the color after application that is greater than or equal to 5.

In an embodiment, the invention provides a cosmetic composition including, in a cosmetically-acceptable medium, at least one multilayer interference pigment and at least one coloring agent that is sensitive to at least one external stimulus (hereinafter called Xchrome), the coverage of the composition being greater than or equal to 25, better 30, the multilayer interference pigment being at a content that imparts to the composition, a difference in color ΔE between the bulk color and the color after application that is greater than or equal to 5.

In an embodiment, the invention provides a cosmetic composition including, in a cosmetically-acceptable medium, at least one multilayer interference pigment and at least one reflecting pigment with metallic reflection, the coverage of the composition being greater than or equal to 25, better 30, the multilayer interference pigment being at a content that imparts to the composition, a difference in color ΔE between the bulk color and the color after application that is greater than or equal to 5.

In an embodiment, the invention provides a cosmetic composition including, in a cosmetically-acceptable medium, at least one multilayer interference pigment and at least one diffracting pigment, the coverage of the composition being greater than or equal to 25, better 30, the multilayer interference pigment being at a content that imparts to the composition, a difference in color ΔE between the bulk color and the color after application that is greater than or equal to 5.

For the purpose of calculating ΔE, the color after application is determined after the composition has been spread on a contrast card, as when measuring coverage.

The coloring agent that is sensitive to at least one external stimulus may be suitable in one state for taking on a bulk color of dominant wavelength that differs from the dominant wavelength of the composition by an amount Δλ of less than 30 nm. Such a characteristic makes it possible for the Xchrome coloring agent and the multilayer interference pigment to be substantially the same color, for example. In a variant, the bulk color of the Xchrome coloring agent in the composition may be colorless, which can be preferable, particularly when said composition is white.

The coloring agent that is sensitive to at least one external stimulus may be suitable in one state for taking on a color of dominant wavelength that differs from the dominant wavelength of the composition, after application to keratinous materials, by an amount Δλ of less than 30 nm. This makes it possible to reinforce the color of the composition after application.

### BRIEF DESCRIPTION OF DRAWINGS

FIG. 1 is a view showing two blocks of cosmetic compositions according to one embodiment.

FIG. 2 is a view showing a cosmetic dispenser to dispense the cosmetic composition disclosed by the embodiment of FIG. 1.

FIG. 3 is a view showing two blocks of cosmetic composition according to an alternative embodiment.

FIG. 4 is a view showing three blocks of cosmetic composition according to an alternative embodiment.

FIG. 5 is a view showing two blocks of cosmetic composition cast or compacted in a cup.

FIG. 6 is a view showing two blocks of cosmetic composition according to an alternative embodiment.

### DESCRIPTION OF PREFERRED EMBODIMENTS

The coloring agent that is sensitive to at least one external stimulus may be colorless in a state that corresponds to the presence of the stimulus or to the absence of the stimulus, for example. This state may exist within the bulk of the composition before application to keratinous materials, or, in a variant, it may exist after application to keratinous material.

Depending on the combinations adopted, the invention offers the possibility of creating multiple effects.

The coloring agent that is sensitive to at least one external stimulus may be in solution in the medium, or it may be dispersed in particulate form in the medium, depending on its nature.

The coloring agent that is sensitive to at least one external stimulus may be a photochromic, thermochromic, solvatochromic, piezochromic, tribochromic, or mechanoluminescent agent, for example.

The invention may include only a single Xchrome coloring agent, or, in a variant, a plurality of Xchrome coloring agents that are sensitive to different kinds of stimuli.

The composition may be anhydrous or aqueous.

Depending on the composition, the color difference ΔE may lie in the range 5 to 30, for example, in particular it may be greater than any integer lying in this range.

The coverage may lie in the range 30 to 70, in particular it may be greater than any integer lying in this range, e.g. greater than or equal to 40.

In multilayer interference pigments, color production by the interference phenomenon is in competition with color production by the phenomenon of absorption by the surface layer of the pigment.

Thus, when the pigment concentration increases sufficiently, the color produced by the interference phenomenon decreases while that produced by absorption increases.

By exploiting this property, the invention makes it possible to observe a variation in color in the composition during application, which imparts a fun aspect in use.

The variation in color on application may be amplified or toned by the presence of the coloring agent that is sensitive to an external stimulus, which coloring agent may itself also change color on application, where appropriate.

The invention also offers novel possibilities with regard to selling cosmetic compositions, making it possible, in the packaging, to take advantage of the variation in color before and after application.

The ratio m₁/m₂ between the content by weight ml of multilayer interference pigment and the content by weight m₂ of Xchrome coloring agent or of reflecting pigment with metallic reflection or of diffracting pigment lies in the range 0.1 to 1.5, for example.

The multilayer interference pigment content by weight may lie in the range 7% to 20%, better in the range 8% to 15%, in particular for a composition that is not powder, e.g. a composition that is liquid or that has been cast.

For a loose- or compact-powder composition, the multilayer interference pigment content lies in the range 40% to 95%, for example, better in the range 50% to 80%.

The bulk color of the composition may be white, while the composition is not exposed to the stimulus. The term “white” should be understood as achromatic in the CIE meaning.

Within its bulk, the composition may have a whiteness index that is greater than or equal to 40.

The composition may not include any coloring agent other than the multilayer interference pigment(s) and:
the coloring agent(s) that are sensitive to an external stimulus or
the reflecting pigment(s) with metallic reflection or
the diffracting pigment(s).

The multilayer interference pigment may comprise at least four layers, for example.

The multilayer interference pigment may include a substrate made of a transparent material.

The multilayer interference pigment may include a substrate made of a material that is rough to a greater or lesser extent, thereby making it possible to impart greater glossiness to the composition.

The substrate is, for example, made of: natural mica that is relatively rough; synthetic mica; alumina; silica; or glass for a smoother surface.

The composition may include two multilayer interference pigments having layers that are made of the same material, with at least one layer of one pigment having a thickness that is different from a corresponding layer of the other pigment, so as to produce different colors.

In embodiments, the composition includes oil(s) at a content of more than 30% of the total weight.

In embodiments, the composition includes wax(es) at a content of more than 10% of the total weight.

In embodiments, the composition includes filler(s) at a content of more than 10% of the total weight.

In another of its aspects, the invention also provides a cosmetic composition that is not powder, e.g. a composition that is liquid (at 25° C.) or that is in stick form, said composition including, in a cosmetically-acceptable medium:
one or more multilayer interference pigments at a content by weight lying in the range 7% to 20%, better in the range 8% to 15% of the total weight; and
one or more coloring agents that is/are sensitive to at least one external stimulus or one or more reflecting pigments with metallic reflection or one or more diffracting pigments, at a content by weight lying in the range 0.1% to 10%.

In another of its aspects, the invention also provides a loose- or compacted-powder composition including, in a cosmetically-acceptable medium:
one or more multilayer interference pigments at a content by weight lying in the range 40% to 95%, better in the range 50% to 80% of the total weight; and
one or more coloring agents that is/are sensitive to at least one external stimulus or one or more reflecting pigments with metallic reflection or one or more diffracting pigments, at a content by weight lying in the range 0.1% to 60%, better 1% to 50%.

In the two above-mentioned aspects, the composition may present one or more of the above-mentioned characteristics, relating for example to the Xchrome coloring agent or to the reflecting pigment with metallic reflection or to the diffracting pigment and to its relationship with the multilayer interference pigment.

Composition with White Color in its Bulk

In an embodiment, the invention provides a solid cosmetic composition, in particular in stick form, comprising in a cosmetically-acceptable medium at least one multilayer interference pigment suitable for conferring a white color to the composition in its bulk, and for giving rise, after application of the composition, to a color variation ΔE of at least 5.

The term “stick” is used to designate a composition in solid form, generally in the form of an elongate block, enabling makeup to be transferred by friction. By way of example, a stick can be obtained by molding or by extrusion.

For the purposes of calculating ΔE, the color after application is determined after the composition has been spread on a contrast card, as for measuring coverage.

In its bulk, the composition may have a whiteness index greater than or equal to 40.

The composition may be anhydrous or aqueous.

Depending on the composition, the difference ΔE may for example lie in the range 5 to 30, in particular it may be greater than any integer lying in that range.

The coverage of the composition may lie in the range 30 to 70, in particular it may be greater than any integer lying in said range, for example it may be greater than or equal to 40.

The amount of multilayer interference pigment may lie in the range 7% to 20% by weight, better 8% to 15%, in particular for a composition that is not in powder form, e.g. that is in stick form.

For a loose or compacted powder composition, the amount of multilayer interference pigment may lie in the range 40% to 95%, better 50% to 80%, for example.

The composition may not comprise any coloring agent other than the multilayer interference pigment(s) and possibly the Xchrome coloring agent, the reflecting pigment or the diffracting pigment.

The multilayer interference pigment may comprise at least four layers, for example.

The multilayer interference pigment may comprise a substrate of a material that is rough to a greater or lesser extent, which can enable a greater or smaller amount of gloss to be imparted to the composition.

By way of example, the substrate may be selected from relatively-rough natural mica, from synthetic mica, from alumina, from silica, or from glass or metal for a surface that is smoother.

The composition may comprise two multilayer interference pigments having layers made up of the same materials, at least one layer of one pigment having thickness that is different from a corresponding layer of the other pigments, so as to produce different colors.

In an embodiment, the invention provides a cosmetic composition comprising, in a cosmetically-acceptable medium, at least one multilayer interference pigment suitable for conferring a white color on the composition in its mass, of whiteness index greater than or equal to 40, the total amount of multilayer interference pigment lying in the range 7% to 20% by weight for a non-powder composition and in the range 40% to 95%, better 40% to 80% for a powder composition.

In an embodiment, the invention also provides packaging comprising:
a composition of white color in its bulk as defined above; and
means for informing the user about the color of the composition after application on keratinous materials (skin, lips, hair, nails, eyelashes, eyebrows). By way of example, that may be done by printing an ink or a varnish, by means of a thin layer of the composition, or by molding or overmolding a material that incorporates the multilayer interference pigment(s).

### Set for the Application of Two Coats

In an embodiment, the invention provides a set comprising:
a first cosmetic composition for applying to keratinous materials, comprising in a cosmetically-acceptable medium at least one multilayer interference pigment, the first composition presenting coverage greater than or equal to 25, better 30, the amount of multilayer interference pigment conferring on the composition a color change ΔE of at least 2, better 5, between its bulk color and its color after application on keratinous materials;
a second composition referred to as a top coat for applying onto the first composition; or
a second composition referred to as a base coat for applying before the first composition on the keratinous materials.

For the purpose of calculating ΔE, the color after application is determined after the composition has been spread on a contrast card, as described in detail below.

The second composition is preferably liquid.

The second composition may include a fatty phase so as to confer gloss to the makeup.

The second composition may be transparent so as to avoid affecting the saturation of the color produced by the first composition.

The second composition need have no solid bodies, so as to avoid diffusing light reflected by the first composition. In a variant, the second composition may include at least one effect pigment, in particular a metallic reflecting pigment, interference pigments, compounds having properties chosen from photochromic, thermochromic, solvatochromic, piezochromic, tribochromic, or mechanoluminescent properties.

The first composition may be anhydrous or aqueous.

Depending on the first composition, the difference ΔE may be greater than any integer value lying in the range 5 to 30.

The coverage of the first composition or of the base coat may be greater than 30, in particular greater than any integer value lying in the range 30 to 70, e.g. greater than or equal to 40.

The percentage by weight of multilayer interference pigment in the first composition may lie in the range 7% to 15%, better 8% to 15%, in particular for a first composition that is not in powder form, e.g. being liquid or in stick form.

With a first composition in powder form, the percentage of multilayer interference pigment may, for example, lie in the range 40% to 95%, better 40% to 80%.

The bulk color of the first composition may be white, i.e. it may be achromatic in the CIE meaning. The bulk color of the base coat composition may have a whiteness index greater than or equal to 40.

The first composition need not include any coloring agent other than the multilayer interference pigment(s) and possibly the Xchrome coloring agent, the reflecting pigment or the diffracting pigment.

The first composition may include two multilayer interference pigments having layers made of the same material, at least one layer of one pigment having a thickness that is different from a corresponding layer of the other pigments so as to produce different colors.

The set may include means for showing the color of the first composition after application or the color of both compositions of the set after application to keratinous materials (skin, lips, nails, eyelashes, eyebrows, hair).

By way of example, that may be done by printing an ink or a varnish, by means of a thin layer of the composition, or a molding or overmolding of a material incorporating the multilayer interference pigment(s). The second composition as a top coat comprises a cosmetically-acceptable medium.

The formulation of this medium is selected in such a manner as to enable it to be applied on the first composition, e.g. for the purpose of conferring gloss and/or improving retention and/or shading an optical effect provided by the first composition.

The second composition as a top coat may comprise a liquid fatty body or a film-forming agent.

The second composition as a top coat may include a colorant or a plurality of active agents and other compounds.

The second composition as a top coat may present any form compatible with application onto the first composition.

When it is desired to obtain gloss, the top coat composition is preferably liquid and transparent, advantageously including a fatty phase.

The second composition may include at least one effect pigment. The pigment should be present in an amount that does not affect observing the color produced by the base coat composition once it has been applied onto keratinous materials.

The composition may be packaged in any receptacle or on any support provided for this purpose. The first and second compositions may be contained, where appropriate, in two compartments of a single packaging device and/or in a single package before first use.

Each composition may be applied by using an optionally flocked applicator, e.g. a foam, an endpiece, a paint brush, a felt, a spatula, a sintered element, a brush, a comb, or an optionally woven fabric.

Application can also be performed using a finger or by placing each composition directly on the substrate that is to be made up, for example by rubbing a stick or by spraying with the help of a piezoelectric or electrostatic device, or by transferring a layer of composition that has previously been deposited on an intermediate substrate.

The first composition can be packaged in a packaging device that makes it possible to see the bulk color of the composition.

The packaging device may be a receptacle having a body that is transparent, at least in part, and/or a packaging device including a stopper member that is transparent, at least in part.

The first composition can be packaged in a device that makes it possible to see both the bulk color of the composition and the color of the composition after application onto keratinous materials.

The first composition can be packaged in a device that includes means that are representative of the color that is revealed on application, e.g. a deposit of a layer of a composition or of an ink or a varnish that includes the same coloring agent as the first composition.

Where appropriate, the first composition or the top coat, second composition may be packaged together with a magnet, making it possible to modify the orientation of the particles of the multilayer interference pigment when said pigment presents non-zero magnetic susceptibility.

### Solid Cosmetic

In an embodiment, the invention provides a solid cosmetic comprising at least:
a first block of a first cosmetic composition containing in a cosmetically-acceptable medium at least one first multilayer interference pigment;
a second block of a second cosmetic composition containing in a cosmetically-acceptable medium at least one second multilayer interference pigment different from the first;

at least one of the first and second compositions being suitable for presenting a first color in its bulk and a second color after application, the first and second colors differing by a difference ΔE greater than or equal to 2, better 5.

For the purposes of calculating ΔE, color after application is determined after the composition has been spread onto a contrast card, as for measuring coverage.

Each of the first and second compositions may be suitable for presenting a first color in its bulk and a second color after it has been applied on keratinous materials, the first and second colors of each composition differing by a difference ΔE greater than or equal to 5.

The first and second blocks may extend over the entire length of a stick or over the entire thickness of the cosmetic when it is cast or compacted in a cup.

The first and second blocks may be concentric or they may be disposed otherwise.

Depending on the compositions, the difference ΔE may lie in the range 5 to 30, for example.

The coverage of one of the compositions may be greater than 25, e.g. lying in the range 30 to 70. The coverage of each composition may be greater than or equal to 25, better 30. Preferably, the coverage of each composition is substantially the same. The coverage of the cosmetic after being cast and then spread so that the compositions of the different blocks mix together may then be greater than or equal to 25, e.g. lying in the range 30 to 70.

In multilayer interference pigments, the production of color by the interference phenomenon is in competition with the production of color by the phenomenon of absorption by the surface layer of the pigment.

Thus, when the pigment concentration increases sufficiently, the color produced by the interference phenomenon diminishes to the advantage of the color produced by absorption.

By making use of this property, the invention makes it possible to observe a variation in the color of at least one of the compositions on application, thus making the cosmetic fun to use.

The invention also provides novel possibilities in terms of commercializing cosmetic compositions, by making it possible for the packaging to make use of this variation in color before and after application.

The invention provides a way of surprising the consumer by making it possible to reveal a plurality of colors on application, in certain circumstances.

The amount of multilayer interference pigment in at least one of the compositions may lie in the range 7% to 20%, by weight, better 8% to 15%.

The color of at least one of the compositions in its bulk may be white. All of the blocks may be white, i.e. achromatic in the CIE meaning, with a whiteness index greater than or equal to 40, for example.

At least one of the compositions or all the compositions need not include any coloring agent other than the multilayer interference pigment. This can apply to all of the compositions of the cosmetic.

The compositions of the cosmetic may comprise two respective multilayer interference pigments having layers made up of the same materials.

At least one layer of a pigment of one of the compositions may have thickness different from a corresponding layer of a pigment of the other composition, so as to produce different colors.

In another of its aspects, the invention also provides packaging comprising:
a cosmetic as defined above; and
means for informing the user about the color of at least one of the compositions of the cosmetic after application, or of a color that is formed by superposing the compositions on application. For example, this may be done by printing an ink or a varnish, by a thin layer of the composition, or by a molding or overmolding of a material incorporating the multilayer interference pigment(s).

In the meaning of the present invention, the term “solid cosmetic” is used to cover a cosmetic having the property of lacking the ability to flow under the action of its own weight, under normal storage conditions.

A solid cosmetic may be a cosmetic of viscosity that is not measurable.

Where appropriate, the solid cosmetic may present a pasty appearance at ambient temperature (25° C.).

At least one of the blocks may present a melting point or thermal transition temperature (e.g. a softening point) greater than 25° C., in particular lying in the range 250° C. to 85° C. or 30° C. to 60° C., and in particular in the range 30° C. to 45° C.

The hardness of at least one of the blocks of composition may, for example, lie in the range 0.001 megapascals (MPa) to 0.5 MPa, in particular in the range 0.005 MPa to 0.4 MPa.

The hardness of the block may be determined by measuring compression force at 20° C. with the help of a texture meter sold under the name TA-XT2i® by the supplier RHEO, fitted with a stainless steel cylinder having a diameter of 2 millimeters (mm) that moves at a measuring speed of 0.01 millimeters per second (mm/s), and penetrating into the composition of the block to a penetration depth of 0.3 mm.

The hardness value is the maximum compression force as measured divided by the area of the cylinder of the texture meter in contact with the composition.

In the particular example of lipstick, hardness can also be measured by the so-called “cheese-wire” method which consists in cutting a stick of lipstick having a diameter of 8.1 mm and in measuring hardness at 20° C. by means of a DFGHS 2 dynamometer from the supplier Indelco-Chatillon moving at a speed of 100 millimeters per minute (mm/min).

The measured hardness is expressed as the shear force (expressed in grams force) needed to cut the stick under such conditions. In this method, the hardness of a solid cosmetic of the invention may lie for example in the range 50 grams (g) to 300 g, e.g. in the range 100 g to 250 g, and for example in the range 120 g to 230 g.

Measuring Coverage

Liquid compositions (at 25° C.)

The term “liquid composition” is used to mean a composition of viscosity that can be measured. A liquid composition can flow under the effect of its own weight.

Coverage of compositions is measured at a finite thickness of 50 micrometers (μm), the liquid compositions being, for example, compositions for application to the lips, in particular liquid lipsticks, liquid lip glosses, and liquid lip balms; nail varnishes; eyeshadows; liquid foundations; mascara; and other liquid makeup that is not for application to the lips.

The composition is spread on matt-black and matt-white contrast cards, e.g. trade name Leneta Form WP1 for the matt-black card and Leneta 1A for the matt-white card.

Application can be performed with an automatic spreader.

The measurements are performed on the compositions spread in this way.

### Solid Compositions (at 25° C.)

Solid compositions are compositions of viscosity that cannot be measured.

Solid compositions are compositions cast in stick form, or they are powders in loose or compacted form.

a) For loose- or compacted-powder solid compositions, the composition is applied using the same contrast cards as above, but that are covered in respective slightly rough transparent adhesive strips, e.g. trade name BLENDERM® by 3M and referenced 15025, which strips are bonded via their adhesive faces on the contrast cards.

The composition is deposited on the adhesive strip in such a manner as to obtain a homogenous deposit of 0.5 micrograms per square centimeter (mg/cm²)±0.02 mg/cm².

In order to perform the deposit, it is possible to use a sponge loaded with composition and mounted on a spreader appliance that causes the sponge to perform predefined movements. By way of example, the sponge is a single-use sponge of “LANCÔME-Photogenic” type, used on its pink side.

b) The compositions in stick form are melted, e.g. at 90° C., then spread in liquid state, with a thickness of 50 μm, onto matt-black or matt-white contrast cards, e.g. as referenced above, but not covered in BLENDERM®. The spreader bar is maintained at the same temperature as the composition, so as to avoid any thermal shock.

### Measurements and Calculations

Reflection spectra are acquired by means of a MINOLTA 3700-d spectrocolorimeter (measurement shape diffuse/8° and observation D65/10°, specular component mode excluded, small opening (CREISS)) on black and white backgrounds, contrast cards optionally being covered in BLENDERM® as indicated above.

The spectra are expressed in calorimetric co-ordinates in the CIELab76 space in the meaning of the CIE in accordance with recommendation 15:2004.

The coverage or contrast ratio is calculated by taking the arithmetic mean of Y on a black background, divided by the mean value of Y on a white background, multiplied by 100.

### Measuring the Bulk Color of the Composition

The bulk color is measured after filling a container that presents a depth that is sufficiently deep to enable the thickness of composition to be considered as being infinite for measurement purposes, e.g. a depth of 3 mm or more.

The L*, a*, and b* co-ordinates are measured with a spectrocolorimeter, e.g. of trade name MINOLTA CM-2002 (D65/10°, specular component mode excluded).

Measuring Color after Application

The color is measured on the dark background of the contrast card, the composition being spread as described above for measuring coverage, but at a thickness of 150 μm instead of 50 μm for liquid compositions and non-powder compositions, in particular compositions in stick form.

The color difference ΔE is calculated as follows:

ΔE=[(a*_{bulk}−a*_{afterapplication})²+(b*_{bulk}−b*_{afterapplication})²+(L*_{bulk}−L*_{afterapplication})²]^{1/2}

When the color difference ΔE varies as a function of the observation angle because of the presence of a goniochromatic agent, the maximum difference is taken as ΔE.

### Measuring Whiteness Index

After measuring color, the whiteness index is calculated at infinite thickness and in accordance with the ASTM E313-05 standard.

### Multilayer Interference Pigment

The expression “multilayer interference pigment” means a pigment that is capable of producing a color by an interference phenomenon between the light rays reflected by a plurality of superposed layers of different refractive indices, in particular a succession of layers of high and low refractive indices. The pigment may comprise a substrate, for example mica, coated with only one coating having a different refraction ratio, for example a coating of TiO₂.

Any multilayer interference pigment may be envisaged.

Any color may be produced by the multilayer interference pigment, e.g. optionally being of dominant wavelength lying in the range 580 nm to 650 nm.

The composition may include a single multilayer interference pigment or a plurality of multilayer interference pigments having different dominant wavelengths.

The multilayer interference pigment may comprise a substrate (also known as a core) that is covered, on at least one face, by one or more layers made of materials and thicknesses that are selected such that a color is produced by interference.

Layers of the interference pigment may optionally surround the substrate which may present an optionally flat shape.

The substrate may include natural mica, synthetic mica, glass, alumina, silica, or even any metal, alloy, or metal oxide. The type of substrate could be selected as a function of the glossiness desired. For example, for a very glossy result, a substrate made of glass or of metal could be preferred.

The interference pigment may include more than four layers of different refractive indices.

The size of the particles of the multilayer interference pigment, given by the mean grain size at half the population, also referred to as D₅₀, lies in the range 1 μm to 2000 μm, for example, better in the range 5 μm to 2000 μm.

The proportion of multilayer interference pigment is greater than 7%, for example, and lies in the range 7% to 20%, for example, for a non-powder, liquid, or cast composition, e.g. a composition in stick form, and in the range 40% to 95%, for example, for a loose- or compacted-powder composition.

The coverage of the composition may essentially be a result of its multilayer interference pigment content. In a variant, at least one diffusing pigment and/or fillers may impart coverage.

Nacres are examples of suitable multilayer interference pigments.

### Nacres

The term “nacre” means colored particles of any form, which may optionally be iridescent, as produced in the shells of certain mollusks, or which are synthesized, and which exhibit a “pearlescent” coloring effect by optical interference.

Examples of nacres that may be mentioned are nacre pigments such as mica titanium coated with iron oxide, mica coated with bismuth oxychloride, mica titanium coated with chromium oxide, mica titanium coated with an organic colorant in particular, and nacre pigments based on bismuth oxychloride.

They may also be particles of mica on the surface of which at least two successive layers of metal oxides and/or organic coloring substances have been superimposed.

The nacres may have a yellow, pink, red, bronze, orangey, brown, gold, and/or coppery color or glint.

Illustrative examples of nacres suitable for being introduced as a multilayer interference pigment and that may be mentioned are gold color nacres, in particular those sold by ENGELHARD under the trade names Brillant gold 20 212G (Timica), Gold 222C (Cloisonne), Sparkle gold (Timica), Gold 4504 (Chromalite), and Monarch gold 233X (Cloisonne); bronze nacres, in particular those sold by MERCK under the trade names Bronze fine (17384) (Colorona) and Bronze (17353) (Colorona), and by ENGELHARD under the trade name Super bronze (Cloisonne); orange nacres in particular those sold by ENGELHARD under the trade names Orange 363C (Cloisonne) and Orange MCR 101 (Cosmica), and by MERCK under the trade names Passion orange (Colorona) and Matte orange (17449) (Microna); brown-tinted nacres in particular those sold by ENGELHARD under the trade names Nu-antique copper 340XB (Cloisonne) and Brown CL4509 (Chromalite); nacres with a copper glint in particular those sold by ENGELHARD under the trade name Copper 340A (Timica); nacres with a red glint, in particular those sold by MERCK under the trade name Sienna fine (17386) (Colorona); nacres with a yellow glint, in particular those sold by ENGELHARD under the trade name Yellow (4502) (Chromalite); red-tinted nacres with gold glints, in particular those sold by ENGELHARD under the trade name Sunstone G012 (Gemtone); pink nacres, in particular those sold by ENGELHARD under the trade name Tan opale G005 (Gemtone); black nacres with a gold glint, in particular those sold by ENGELHARD under the trade name Nu antique bronze 240 AB (Timica); blue nacres, in particular those sold by MERCK under the trade name Matte blue (17433) (Microna); white nacres with silvery glints, in particular those sold by MERCK under the trade name Xirona Silver; and orange-pink green-gold highlight nacres in particular those sold by MERCK under the trade names Indian summer (Xirona); and mixtures thereof.

By way of example, multilayer interference pigments presenting magnetic properties are those sold under the trade names: COLORONA BLACKSTAR BLUE, COLORONA BLACKSTAR GREEN, COLORONA BLACKSTAR GOLD, COLORONA BLACKSTAR RED, CLOISONNE NU ANTIQUE SUPER GREEN, MICRONA MATTE BLACK (17437), MICA BLACK (17260), COLORONA PATINA SILVER (17289), and COLORONA PATINA GOLD (117288) by MERCK; or indeed: FLAMENCO TWILIGHT RED, FLAMENCO 25 TWILIGHT GREEN, FLAMENCO TWILIGHT GOLD, FLAMENCO TWILIGHT BLUE, TIMICA NU ANTIQUE SILVER 110 AB, TIMICA NU ANTIQUE GOLD 212 GB, TIMICA NU-ANTIQUE COPPER 340 AB, TIMICA NU ANTIQUE BRONZE 240 AB, CLOISONNE NU ANTIQUE GREEN 828 CB, CLOISONNE NU ANTIQUE BLUE 626 CB, GEMTONE MOONSTONE G 004, CLOISONNE NU ANTIQUE RED 424, CHROMA-LITE BLACK (4498), CLOISONNE NU ANTIQUE ROUGE FLAMBE (code 440 XB), CLOISONNE NU ANTIQUE BRONZE (240 XB), CLOISONNE NU ANTIQUE GOLD (222 CB), and CLOISONNE NU ANTIQUE COPPER (340 XB) by ENGELHARD.

The multilayer interference pigment may be chosen from those giving a bulk white color to the composition, for example the nacres sold by ENGELHARD under the trade names SPARKLE 110P (Timica), Flamenco blue (Flamenco), Flamenco green (Flamenco), Flamenco red (Flamenco), Flamenco violet (Flamenco), Flamenco orange (Flamenco), Silkbalnc 110W (Timica), Extra large sparkle (Timica), Flamenco sparkle Gold (Flamenco), Flamenco sparkle green (Flamenco), Flamenco sparkle orange (Flamenco), Flamenco sparkle blue (Flamenco), Flamenco sparkle violet (Flamenco), Flamenco sparkle red (Flamenco), Flamenco summit gold (Flamenco); the nacres sold by MERCK under the trade name Silk blue (Timiron), Silk green (Timiron), Silk red (Timiron), Super red (Timiron), Super green (Timiron), Super blue (Timiron), Artic Silver (Timiron), splendid copper (Timiron), Splendid Violet (Timiron); the nacres sold by ECKART under the trade name Prestige Silver (Prestige), Prestige Silver Star (Prestige), Prestige Gold (Prestige), Prestige soft gold (Prestige), Prestige silk green (Prestige), Prestige silk lilac (Prestige), Prestige silk blue (Prestige), Prestige silk red (Prestige).

The multilayer interference pigment may be deprived of a coating of a colored material, for example chosen from FeOOH, Fe₂O₃, Cr₂O₃, TiO₂-x, TiOxNy, CrPO₄, KFe[Fe(CN)6], Fe₃O₄, TiO, TiN, FeTiO₃, C, Ag, Au, Fe, Mo, Cr, W.

The multilayer interference pigment may for example only comprise on the substrate one or several layers of materials chosen from TiO₂ (rutile or anatase), ZrO₂, SnO₂, SiO₂.

The multilayer interference pigment may also be selected from the reflective interference particles.

### Reflective Interference Particles

These particles may be selected from particles of synthetic substrate at least partially coated with at least one layer of at least one metal oxide selected, for example, from oxides of titanium, in particular TiO₂, of iron, in particular Fe₂O₃, of tin, or of chromium, barium sulfate, and the following materials: MgF₂, CrF₃, ZnS, ZnSe, SiO₂, Al₂O₃, MgO, Y₂O₃, SeO₃, SiO, HfO₂, ZrO₂, CeO₂, Nb₂O₅, Ta₂O₅, MoS₂, and mixtures or alloys thereof.

Examples of such particles that may be mentioned are particles comprising a substrate of synthetic mica coated with titanium dioxide, or glass particles coated either with brown iron oxide, titanium oxide, tin oxide, or one mixture thereof such as those sold under the trade name REFLECKS® by ENGELHARD.

The multilayer interference pigment may also be a goniochromatic pigment.

### Goniochromatic Pigment

The term “goniochromatic pigment” as used in the context of the present invention means a pigment that makes it possible, when the composition is spread on a substrate, to obtain a color path in the a*b* plane of the 1976 CIE color space which corresponds to a variation Dh° of the hue angle h° of at least 20° when the angle of observation is varied relative to the normal in the range 0 to 80° for light at an angle of incidence of 45°.

By way of example, the color path may be measured by means of a spectrogonioreflectometer, from INSTRUMENT SYSTEMS and referenced GON 360 GONIOMETER, after the composition has been spread in the fluid state to a thickness of 300 μm by means of an automatic spreader on a contrast card from ERICHSEN and referenced Typ 24/5, the measurements being performed on the black background of the card.

The goniochromatic pigment may, for example, be selected from multilayer interference structures and liquid crystal coloring agents.

By way of example, a multilayer structure may comprise at least two layers, each layer being produced, for example, from at least one material selected from the group constituted by the following materials: MgF₂, CeF₃, ZnS, ZnSe, Si, SiO₂, Ge, Te, Fe₂O₃, Pt, Va, Al₂O₃, MgO, Y₂O₃, S₂O₃, SiO, HfO₂, ZrO₂, CeO₂, Nb₂O₅, Ta₂O₅, TiO₂, Ag, Al, Au, Cu, Rb, Ti, Ta, W, Zn, MoS₂, cryolite, alloys, polymers, and combinations thereof.

The multilayer structure may optionally be symmetrical with respect to a central layer as regards the chemical nature of the stacked layers.

Depending on the thickness and nature of the various layers, different effects are obtained.

Examples of symmetrical multilayer interference structures are as follows: Fe₂O₃/SiO₂/Fe₂O₃/SiO₂/Fe₂O₃, a pigment having this structure being sold under the trade name SICOPEARL by BASF; MoS₂/SiO₂/mica-oxide/SiO₂/MoS₂; Fe₂O₃/SiO₂/mica-oxide/SiO₂/Fe₂O₃; TiO₂/SiO₂/TiO₂ and TiO₂/Al₂O₃/TiO₂, pigments having these structures being sold under the trade name XIRONA by MERCK (Darmstadt).

By way of example, liquid crystal coloring agents comprise silicones, or cellulose ethers onto which mesomorphic groups have been grafted. Examples of suitable liquid crystal goniochromatic particles are those sold by CHENIX, and those sold under the trade name HELICONE® HC by WACKER.

Suitable goniochromatic pigments are some nacres; pigments having effects on synthetic substrates, in particular alumina, silica, borosilicate, iron oxide, or aluminum type substrates; or interference flakes coming from a polyterephthalate film.

The material may further contain dispersed goniochromatic fibers. Such fibers could present a length that is less than 80 μm, for example.

### Xchrome Coloring Agent

They may be photochromic coloring agents.

### Photochromic Coloring Agents

In general, a photochromic coloring agent is a coloring agent having the property of changing hue when it is illuminated or not illuminated by ultraviolet light and to re-establish its initial color when it is no longer illuminated or is illuminated by a light, or passes from a non-colored state to a colored state and vice versa. In other words, such an agent has different hues depending on whether it is illuminated with light containing a certain quantity of UV radiation.

In the presence of a low level of light, the photochromic coloring agent may take on a substantially non colored state.

In the presence of strong illumination, the photochromic coloring agent may take on a colored state, for example a dark hue or a color that has substantially the same dominant wavelength as the multilayer interference pigment, thereby making it possible to make the multilayer interference pigment appear less brilliant than in the presence of low level illumination. This effect can surprise the observer and render the makeup particularly attractive.

The photochromic coloring agent may have a difference ΔE of at least 5. ΔE designates the difference in hue observed in the photochromic substance between its excited state, i.e. in the presence of UV radiation, and its non-excited state, i.e. in the absence of UV radiation.

Reference may usefully be made to examples of photochromic agents described in United States patent application US-A-2004/0228818 the contents of which are hereby incorporated by reference, in particular those with a ΔE of more than 5, as determined using the test presented in this document.

Examples of photochromic coloring agents are naphthopyrane derivatives of the 2H-naphtho-[2,1-b]-pyrane type with formula (I) or 3H-naphtho-[2,1-b]-pyrane type with formula (II):

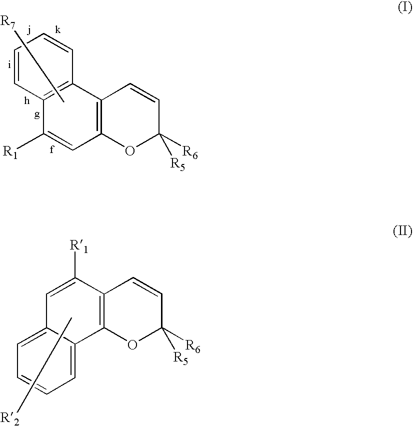

in which:

R₁ represents:

(i) a hydrogen atom;

(ii) a linear, branched, or cyclic, saturated or unsaturated hydrocarbon group containing 1 to 30 carbon atoms, optionally comprising 1 to 5 heteroatoms selected from N, O, S, Si, and P, and/or optionally halogenated or perhalogenated;

(iii) a hydrocarbon cycle formed with one of the “f” or “gh” bonds and the radical R₇; or

(iv) a group selected from —COOR₄, —C(O)NR₂R₃, —NR₂R₃, —OR₄ and —SR₄, in which:

R₂ and R₃ either independently represent a linear, branched, or cyclic, saturated or unsaturated hydrocarbon group containing 1 to 20 carbon atoms, optionally comprising 1 to 5 heteroatoms selected from N, O, S, Si, and P;

or, taken together with the nitrogen atom to which they are bonded, form a saturated or unsaturated hydrocarbon heterocycle containing 3 to 10 carbon atoms and optionally 1 to 5 other heteroatoms selected from N, O, S, Si and P, said cycle optionally being substituted with at least one linear, branched or cyclic, saturated or unsaturated hydrocarbon radical containing 1 to 20 carbon atoms optionally comprising 1 to 5 heteroatoms selected from N, O, S, Si, and P;

R₄ represents a linear, branched or cyclic, saturated or unsaturated hydrocarbon group containing 1 to 20 carbon atoms and/or optionally comprising 1 to 5 heteroatoms selected from N, O, S, Si, and P;

R₅ and R₆ independently represent a group selected from:

(i) saturated cyclic aminoaryl groups with formula (IIA) or (IIB):

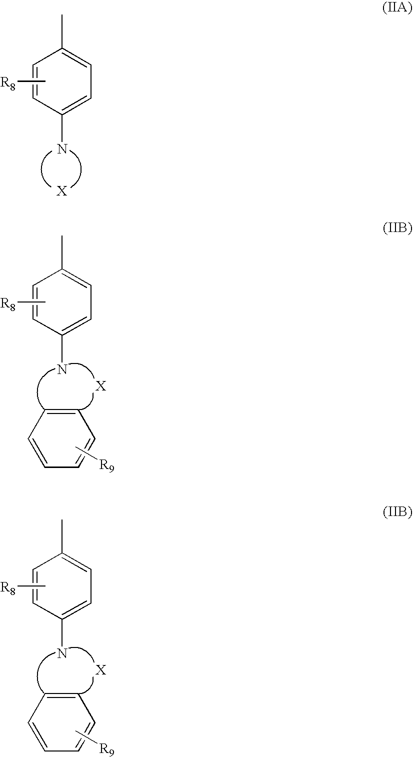

in which the cycle comprising N and X is a saturated cycle which contains a total of 3 to 30 atoms including nitrogen, the remainder being carbon atoms and/or heteroatoms selected from O, S, Si, P, and/or groups selected from —NH and —NR in which R represents a linear, branched, or cyclic, saturated or unsaturated hydrocarbon radical containing 1 to 20 carbon atoms, optionally comprising 1 to 5 heteroatoms selected from N, O, S, Si, and P;

(ii) indolinoaryl groups with formula (III):

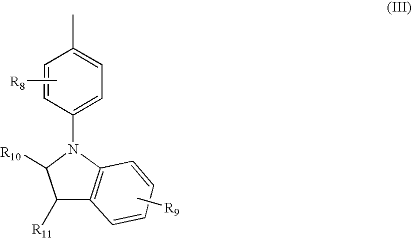

in which R₁₀ and R₁₁ independently represent a group selected from (i) linear, branched, or cyclic, saturated or unsaturated hydrocarbon groups containing 1 to 30 carbon atoms, optionally comprising 1 to 5 heteroatoms selected from N, O, S, Si, and P, and/or optionally halogenated or perhalogenated; (ii) halogen atoms; (iii) —CN (nitrile), —COOH (carboxylate), —NO₂ (nitro) groups; (iv) a hydrogen atom; (v) a group selected from —(O)NR₂R₃, —NR₂R₃, —OR₄ and —SR₄ in which R₂, R₃ and R₄ have the meanings given above; (vi) radicals R₁₀ and R₁₁ may together form a saturated or unsaturated hydrocarbon cycle having a total of 5 to 8 atoms (including the atoms of the indoline cycle), said atoms being selected from C, O, S and/or NR in which R represents H or a linear, branched or cyclic, saturated or unsaturated hydrocarbon radical containing 1 to 20 carbon atoms, optionally comprising 1 to 5 heteroatoms selected from N, O, S, Si, and P;

(iii) groups with formula (IV):

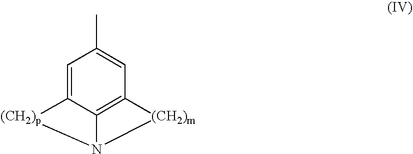

in which m and p are independently integers from 2 to 5;

(iv) unsaturated cyclic aminoaryl groups with formulae (VA), (VB), or (VC):

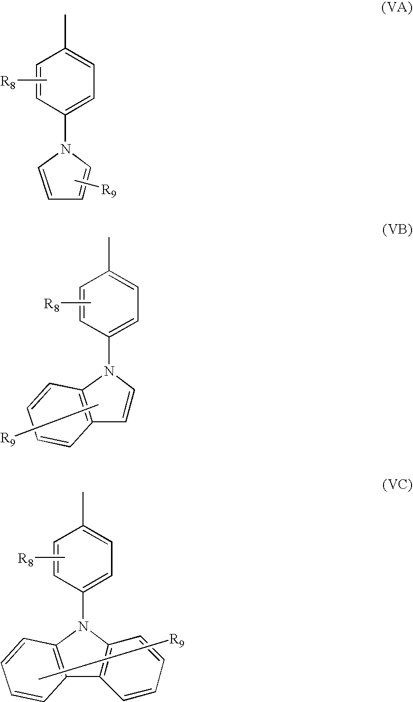

in which R₈ and R₉, independently represent a group selected from (i) linear, branched, or cyclic, saturated or unsaturated hydrocarbon groups containing 1 to 30 carbon atoms, optionally comprising 1 to 5 heteroatoms selected from N, O, S, Si, and P, and/or optionally halogenated or perhalogenated; (ii) halogen atoms; (iii) —CN (nitrile), —COOH (carboxylate), —NO₂ (nitro) groups; (iv) a hydrogen atom; (v) a group selected from —C(O)NR₂R₃, —NR₂R₃, —OR₄, and —SR₄, in which R₂, R₃ and R₄ have the meanings given above;

(v) a linear, branched or cyclic, saturated or unsaturated hydrocarbon group containing 1 to 30 carbon atoms optionally comprising 1 to 5 heteroatoms selected from N, O, S, Si and P; and in particular a group selected from —CONR₂R₃, —C₆H₄—NR₂R₃, and —C₆H₄—OR₄ in which R₂, R₃ and R₄ have the meanings given above;

R₇ represents a group selected from:

(i) linear, branched or cyclic, saturated or unsaturated hydrocarbon groups containing 1 to 30 carbon atoms, optionally comprising 1 to 5 heteroatoms selected from N, O, S, Si, and P, and/or optionally halogenated or perhalogenated;

(ii) halogen atoms;

(iii) —CN (nitrile), —COOH (carboxylate), —NO₂ (nitro); —N═N-(azo); ═NH (imino); —CONH₂ (amide) groups;

(iv) a hydrogen atom;

(v) a group selected from —C(O)NR₂R₃, —NR₂R₃, —OR₄ and —SR₄ in which R₂, R₃ and R₄ have the meanings given above;

(vi) radical R₇ may also form, with one of the “i”, “j”, “k”, or “g,h” bonds taken with radical R₁, or “f” taken with radical R₁, a saturated hydrocarbon cycle containing a total of 3 to 8 carbon atoms, optionally comprising 1 to 5 heteroatoms selected from N, O, S, Si, and P;

R′₁ represents a group selected from:

(i) a hydrogen atom;

(ii) a linear, branched or cyclic, saturated or unsaturated hydrocarbon group containing 1 to 30 carbon atoms optionally comprising 1 to 5 heteroatoms selected from N, O, S, Si, and P, and/or optionally halogenated or perhalogenated;

(iii) a group selected from —C(O)NR₂R₃, —NR₂R₃, —OR₄, and —SR₄, in which R₂, R₃ and R₄ have the meanings given above;

R′₂ represents a group selected from:

(i) linear, branched or cyclic, saturated or unsaturated hydrocarbon groups containing 1 to 30 carbon atoms, optionally comprising 1 to 5 heteroatoms selected from N, O, S, Si and P, and/or optionally halogenated or perhalogenated;

(ii) halogen atoms;

(iii) —CN (nitrile), —COOH (carboxylate), —NO₂ (nitro); —N═N-(azo); ═NH (imino); —CONH₂ (amide) groups;

(iv) a hydrogen atom;

(v) a group selected from —C(O)NR₂R₃, —NR₂R₃, —OR₄ and —SR₄ in which R₂, R₃ and R₄ have the meanings given above.

Further examples of photochromic agents that may be mentioned are diarylethene with formula:

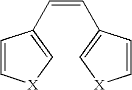

and its derivatives;
dihydroazulene/vinylhepta fulvene, with formula:

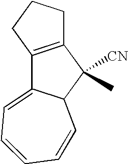

and its derivatives;
spyronaphthoxazine, with formula:

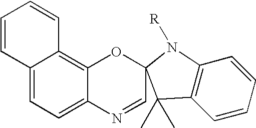

and its derivatives.

The photochromic agent may be an organic or an inorganic compound. When the photochromic agent is an organic compound, the color change may generally be more rapid and intense.

Examples of photochromic agents that may be mentioned are Photosol® from PPG, which reversibly changes color when activated by UV radiation with a wavelength in the range 300 nm to 360 nm, Reversacol® from J. ROBINSON and Photogenica® from CATALYST & CHEMICALS.

### Thermochromic Agents

A thermochromic agent is a pigment or colorant that can change color as a function of temperature.

The thermochromic agent has, for example, a color that is lost when the temperature exceeds a certain value, for example about 15° C. or about 30° C., depending on the nature of the thermochromic agent.

The thermochromic agent may comprise capsules of a polymer containing a solvent, that solvent, depending on whether it is in the molten state or otherwise, allowing compounds to come into contact and modify the light absorption properties.

The color change may be reversible.

As an example, it is possible to use the thermochromic agent sold under the trade name Kromafast® Yellow5GX 02 by KROMACHEM LTD, or Chromazone® as a powder or a dispersion, or Thermobatch® or Thermostar®, from CHROMAZONE.

### Piezochromic and Tribochromic Agents

A piezochromic agent can change color in the presence of a mechanical force.

An example of a piezochromic agent that may be mentioned is diphenylflavylene.

A tribochromic agent can change color in the presence of a mechanical force in a manner which is more durable than with piezochromic agents.

Reference may be made to International patent application WO-A-94/26729, the contents of which are hereby incorporated by reference.

### Mechanoluminescent Agents

These agents are capable of emitting light when they receive a mechanical stress such as compression, shear, or friction.

The mechanoluminescent agent is preferably in the form of a particle which is insoluble in the cosmetic medium. The mean particle size is, for example, in the range 0.01 μm to 50 μm preferably in the range 0.1 μm and 10 μm.

Mechanoluminescent materials that may be mentioned are as follows:

a) complexes and chelates of lanthanides such as those described in publications U.S. Pat. No. 6,071,632, US-A-2002/0015965 and WO-A-09/016,429, the contents of which are hereby incorporated by reference. The rare earths are preferably selected from europium, terbium, samarium, and dysprosium. In those materials, diketones are used as the ligand for the trivalent lanthanide salts.

b) aluminates, silicates and aluminosilicates doped with rare earth ions such as those described in U.S. Pat. No. 6,280,655, EP-A-0 1 318 184, JP-A-2002/194349, JP-A-2004/59746, the contents of which are hereby incorporated by reference, in particular (Sr,Mg,Ba,Zn,Ca) Al₂O₄, (SrLa,SrY)Al₃O₇, (Sr₂,SrMg,SrCa,SrBa)Al₆O₁₁, Sr₂(Mg,Al)(Al,Si)SiO₇, Sr(Zn,Mn,Fe,Mg)Si₂O₆. The elements shown in parentheses are partially or entirely interchangeable. Rare earth ions such as cerium, europium, samarium, neodymium, gadolinium, dysprosium, and terbium may be used, alone or as a mixture. Europium and dysprosium are preferred;

c) zinc sulfide, manganese sulfide, copper sulfide, cadmium sulfide or zinc oxide, optionally doped with transition metal ions or rare earth ions as described in the publications U.S. Pat. No. 6,117,574 and JP-A-2004/43656 the contents of which are incorporated by reference. Preferred transition metal ions are copper or manganese. Preferred rare earth ions are europium or cerium. Of these materials, ZnS:Mn is preferred.

The materials listed under b) and c) may be synthesized by a solid phase reaction involving dry mixing followed by heat treatment and high temperature sintering, or by a sol-gel process followed by drying, heating and sintering. As an example, the sintering temperature is more than 1000° C.

The materials listed under b) are preferred. Of these, SrAl₂O₄ and SrMgAl₁₀O₁₇ doped with rare metals are preferred.

The mechanoluminescent pigments SrAl₂O₄ doped with rare metal ions are sold with reference TAIKO-Ml-1 by TAIKO Refractories Co., Ltd. The particles of this pigment have a diameter lying in the range 5 μm to 10 μm and a green luminescence under a weak mechanical stress.

### Solvatochromic Agents

A solvatochromic agent can change color in the presence of solvents. DC Red 27 is an example, this compound having an absence of color in an anhydrous formulation; adding water reveals a pink color.

Reflecting Pigment with Metallic Reflection

The reflecting pigment with metallic reflection is not a multilayer interference pigment in the meaning of the invention.

The size of the reflecting particles with metallic reflection in the reflecting pigments with metallic reflection may lie for example in the range 10 micrometers (μm) to 500 μm, and preferably lies in the range 10 μm to 150 μm. The size may be greater than or equal to 40 μm in order to enable it to be perceived visually.

The reflecting particles with metallic reflection may be in flakes or they may be globular, in particular spherical, optionally elongate, with a large form factor, where appropriate. When in the form of flakes, that can enable reflection to be more directional. In contrast, a substantially spherical shape can serve to provide reflection that is more diffuse.

By way of example, the reflecting particles with metallic reflection include at least one electrically conductive layer on the surface, formed by at least one optionally doped metal or metal oxide.

The reflecting particles with metallic reflection may present on the surface at least a protective layer that is sufficiently transparent to conserve the looked-for metallic reflection effect of the particle.

The reflecting particles with metallic reflection, regardless of their shape, may optionally present a multilayer structure, and with a multilayer structure, they may include at least one layer of an optionally doped metal or alloy. The layer may be of uniform thickness.

When the reflecting particles do not present a multilayer structure, they may be constituted, for example, by at least one optionally doped metal or metal alloy.

When the reflecting particles with metallic reflection presents a multilayer structure, they may have a substrate that is natural or synthetic, in particular a synthetic substrate that is coated at least in part by at least one layer of at least one metal, alloy, or metal oxide.

By way of example, this layer is the outer layer of the structure. The substrate may be made of a single material or a plurality of materials, that may be organic and/or inorganic. More particularly, the substrate may be selected from glasses, ceramics, graphite, metal oxides, aluminas, silicas, silicates, in particular aluminosilicates and borosilicates, synthetic mica, and mixtures thereof, this list not being limiting.

As examples of reflecting particles with metallic reflection comprising an inorganic substrate coated in a layer of metal, mention can be made of particles comprising a borosilicate substrate coated in silver. Particles having a glass substrate coated in silver and in the form of flakes are sold under the name MICROGLASS METASHINE REFSX 2025 PS by the supplier TOYAL. Particles with a glass substrate coated in an alloy of nickel-chromium-molybdenum are sold under the name CRYSTAL STAR GF 550, GF 2525 by the same supplier.

As examples of reflecting particles with metallic reflection presenting on the surface a metal compound or including at least one metal compound, possibly coated, mention can be made of the particles made available under the names METASHINE® ME 2040 PS, METASHINE® MC5090 PS, or METASHINES MC280GP (2523) by the supplier NIPPON SHEET GLASS; SPHERICAL SILVER POWDER® DC 100, SILVER FLAKE® JV6, or GOLD POWDER® A1570 from the supplier ENGELHARD; STARLIGHT REFLECTIONS FXM® from the supplier ENERGY STRATEGY ASSOCIATES INC.; BRIGHT SILVER® 1 E 0.008X0.008 from the supplier MEADOWBROOK INVENTIONS; ULTRAMIN® (ALUMINIUM POUDRE FINE LIVING), and COSMETIC METALLIC POWDER VISIONAIRE BRIGHT SILVER SEA®, COSMETIC METALLIC POWDER VISIONAIRE NATURAL GOLD® (60314), or COSMETIC METALLIC POWDER VISIONAIRE HONEY® (60316) from the supplier ECKART.

The reflecting particles with metallic reflection may reflect the visible spectrum in substantially uniform manner, as happens for example with particles coated in a metal such as silver or aluminum, or otherwise, which can then lead for example to a metallic reflection having a hue that is not neutral, e.g. yellow, pink, red, bronze, orangey, brown, and/or coppery, e.g. depending on the nature of the surface metal compound.

The reflecting particles with metallic reflection may be present in the composition in an amount lying in the range 0.1% to 60% relative to the total weight of the first composition, and in particular in the range 1% to 30% by weight, e.g. in the range 3% to 10% by weight.

The amount of reflecting particles with metallic reflection should be sufficiently small to avoid impeding the production of color by the multilayer interference pigment.

The reflecting pigment with metallic reflection may contribute to obtaining the looked-for coverage.

### Diffracting Pigment

The term “diffracting pigment” as used in the present invention means a pigment which is capable of producing a color variation depending on the angle of observation when illuminated with white light due to the presence of a structure which diffracts light.

Such a pigment is also sometimes termed a holographic pigment or rainbow effect pigment.

A diffracting pigment may comprise a diffraction matrix capable, for example, of diffracting an incident ray of monochromatic light in predetermined directions.

The diffraction matrix may comprise a periodic motif, in particular a line, the distance between two adjacent motifs being of the same order of magnitude as the wavelength of the incident light.

When the incident light is polychromatic, the diffraction matrix separates the various spectral components of the light and produces a rainbow effect. Concerning the structure of diffracting pigments, reference should be made to the article “Pigments Exhibiting Diffractive Effects” by Alberto Argoitia and Matt Witzman, 2002, Society of Vacuum Coaters, 45^{th} Annual Technical Conference Proceedings, 2002, the contents of which are hereby incorporated by reference.

The diffracting pigment may be produced with motifs having different profiles, in particular triangular, symmetrical or unsymmetrical, crenellated, with a constant or non constant width, sinusoidal, or stepped.

The spatial frequency of the matrix and the motif depth will be selected as a function of the desired degree of separation of the various orders. As an example, the frequency may lie in the range 500 to 3000 lines per mm.

Preferably, the particles of diffracting pigment each have a flattened form, in particular in the form of a platelet. The same pigment particle may comprise two crossed diffraction matrices, which may or may not be perpendicular, and may or may not have the same spacing.

The diffracting pigment may have a multi-layered structure comprising a layer of reflective material, covered on at least one side by a layer of a dielectric material. This layer may provide the diffracting pigment with better rigidity and durability. The dielectric material may thus, for example, be selected from the following materials: MgF₂, SiO₂, Al₂O₃, AlF₃, CeF₃, LaF₃, NdF₃, SmF₂, BaF₂, CaF₂, LiF, and combinations thereof.

The reflective material may, for example, be selected from metals and their alloys and also from non-metallic reflective materials. Metals which may be mentioned include Al, Ag, Cu, Au, Pt, Sn, Ti, Pd, Ni, Co, Rd, Nb, Cr, and their materials, combinations or alloys. Such a reflective material may alone constitute the diffracting pigment which is then a monolayer.

In a variation, the diffracting pigment may comprise a multi-layered structure comprising a substrate of a dielectric material covered on at least one side by a reflective layer, or even completely encapsulating the substrate.

A layer of a dielectric material may also cover the reflective layer or layers. The dielectric material used is thus preferably inorganic and may, for example, be selected from metal fluorides, metal oxides, metal sulfides, metal nitrides, metal carbides and combinations thereof. The dielectric material may be in the crystalline, semi-crystalline or amorphous state. The dielectric material in this configuration may, for example, be selected from the following materials: MgF₂, SiO, SiO₂, Al₂O₃, TiO₂, WO, AlN, BN, B₄C, WC, TiC, TiN, N₄Si₃, ZnS, glass particles, diamond type carbons, and combinations thereof. In a variation, the diffracting pigment may be composed of a dielectric or preformed ceramic material such as a mineral in natural lamellae, for example mica peroskovite or talc, or synthetic lamellae formed from glass, alumina, SiO₂, carbon, an iron oxide/mica, mica coated with BN, BC, graphite, bismuth oxychloride, and combinations thereof.

Instead of a layer of a dielectric material, other materials which improve the mechanical properties may be suitable. Such materials may include silicone, metal silicides, semiconductor materials formed from elements from groups III, IV, and V, metals having a body-centered cubic crystal structure, Cermet compositions or materials, semiconductor glasses, and their various combinations. The diffracting pigment used may in particular be selected from those described in United States patent application US-2003/0031870 published on Feb. 13, 2003. A diffracting pigment may, for example, comprise the following structure: MgF₂/Al/MgF₂, a diffracting pigment having that structure being sold under the trade name SPECTRAFLAIR 1400 Pigment Silver by FLEX PRODUCTS, or SPECTRFLAIR 1400 Pigment Silver FG. The proportion by weight of MgF₂ may be in the range 80% to 95% of the total weight of the pigment. Other diffracting pigments are sold under the trade names METALURE® PRISMATIC by ECKART®.

Other possible structures are Fe/Al/Fe or Al/Fe/Al.

The dimension of the diffracting pigment may, for example, be in the range 5 μm to 200 μm, better in the range 5 μm to 100 μm, for example in the range 5 μm to 30 μm. The thickness of the particles of diffracting pigment may be 3 μm or less, preferably 2 μm, for example of the order of 1 μm.

### Cosmetically-Acceptable Medium

The term “cosmetically-acceptable medium” means a medium that is not toxic and that is suitable for application to the keratinous materials of human beings.

The cosmetically-acceptable medium is adapted to the nature of the substrate on which the composition is to be applied, and also to the form in which the composition is to be packaged.

The composition of the invention may comprise an aqueous medium and/or a fatty phase that is optionally anhydrous.

### Aqueous or Fatty Phase

The composition may comprise water or a mixture of water and hydrophilic organic solvents, such as alcohols, in particular linear or branched lower monoalcohols containing 2 to 5 carbon atoms, such as ethanol, isopropanol or n-propanol, polyols such as glycerine, diglycerine, propylene glycol, sorbitol, penthylene glycol, or polyethylene glycols.

The hydrophilic phase may also contain hydrophilic C₂ ethers and C₂-C₄ aldehydes.

The water or mixture of water and hydrophilic organic solvents may be present in the composition of the invention at a content lying in the range 0 to 90%, in particular in the range 0.1% to 90% by weight relative to the total composition weight, and preferably in the range 0 to 60% by weight, more particularly in the range 0.1% to 60% by weight.

The composition may also include a fatty phase, in particular constituted by fat that is liquid at 25° C. and possibly fat that is solid at ambient temperature, such as waxes, pasty fats, gums, and mixtures thereof.

Fats that are liquid at ambient temperature, usually termed “oils”, that are used in the invention and that may be mentioned are amongst others: hydrocarbon-containing vegetable oils such as liquid fatty acid triglycerides containing 4 to 10 carbon atoms, for example heptanoic or octanoic acid triglycerides, or even sunflower, corn, soybean, grapeseed, sesame seed, apricot kernel, macadamia nut, castor, or avocado stone oil, caprylic/capric acid triglycerides, jojoba oil, shea nut butter oil; linear or branched hydrocarbons of mineral or synthetic origin, such as paraffin oils, in particular C₈-C₁₆ isoparaffins such as isododecane, isodecane, isohexadecane, Vaseline, polydecenes, hydrogenated polyisobutene such as Parleam®; squalane; synthesized esters and ethers, in particular fatty acids such as Purcellin oil, isopropyl myristate, 2-ethylhexyl palmitate, 2-octyldodecyl stearate, 2-octyldodecyl erucate, isostearyl isostearate; hydroxylated esters such as isostearyl lactate, octylhydroxystearate, octyldodecyl hydroxystearate, diisostearylmalate, triisocetyl citrate, fatty alcohol heptanoates, octanoates, or decanoates; polyol esters such as propylene glycol dioctanoate, neopentylglycol diheptanoate, diethyleneglycol diisononanoate; and pentaerythritol esters; fatty alcohols containing 12 to 26 carbon atoms, such as octyldodecanol, 2-butyloctanol, 2-hexyldecanol, 2-undecylpentadecanol, or oleic alcohol; fluorinated hydrocarbon and/or silicone oils; silicone oils such as volatile or non-volatile, linear or cyclic polymethyl-siloxanes (PDMS) which may be liquid or pasty at ambient temperature, such as cyclomethicones or dimethicones, optionally comprising a phenyl group, such as phenyl trimethicones, phenyltrimethylsiloxydiphenyl siloxanes, diphenylmethyldimethyl-trisiloxanes, diphenyl dimethicones, phenyl dimethicones, polymethylphenyl-siloxanes; and mixtures thereof.

The oils may be present at a content lying in the range 0.01% to 90% relative to the total composition weight.

The composition of the invention may also include one or more physiologically-acceptable organic solvents. The solvent(s), which may be lipophilic, may be present at a content lying in the range 0 to 90%, better in the range 0 to 60% by weight relative to the total composition weight, and still better in the range 0.1% to 30%.

The medium may include a liquid organic phase in which water is dispersed or emulsified.

The composition may also have a continuous fatty phase which may contain less than 5% water, in particular less than 1% water relative to its total weight, and in particular it may be in the anhydrous form.

### Film-Forming Agents

The medium may comprise a film-forming agent, in particular a film-forming polymer.

The term “film-forming agent” is used to mean an agent suitable, on its own or in the presence of an auxiliary film-forming agent, for forming a macroscopically continuous film that adheres on keratinous materials, and preferably a film that is cohesive, and better still a film presenting cohesion and mechanical properties that are such that such film can be isolated and handled in isolation, e.g. when said film is formed by casting onto a non-stick surface such as a Teflon or silicone surface.

The composition may include an aqueous phase and the film-forming polymer may be present in the aqueous phase. The film-forming polymer could be a polymer in dispersion or in solution.

The composition may include an oily phase and the film-forming polymer may be present in the oily phase. The polymer could thus be in dispersion or in solution.

Suitable film-forming polymers that may be mentioned include synthetic polymers, of the radical or polycondensate type, natural polymers, and mixtures thereof.

Radical type film-forming polymers may in particular be vinyl polymers or copolymers, in particular acrylic polymers.

Examples of film-forming polycondensates that can be mentioned include polyurethanes, polyesters, polyester amides, polyamides, epoxyester resins, and polyureas.

Polyesters may be obtained in known manner by polycondensation of dicarboxylic acids with polyols, in particular diols.

Polyester amides may be obtained in similar manner to polyesters, by polycondensation of diacids with diamines or amino alcohols.

By way of example of a liposoluble film-forming polymer, mention can be made of copolymers of vinyl ester (the vinyl group being directly connected to the oxygen atom of the ester group and the vinyl ester having a saturated, linear, or branched hydrocarbon radical with one to 19 carbon atoms bonded to the carbonyl of the ester group) and at least one other monomer which may be: a vinyl ester (different from the already-present vinyl ester); an α-olefin having eight to 28 carbon atoms); an alkyl vinyl ether in which the alkyl group has two to 18 carbon atoms); or an allyl or methallyl ester (having a saturated, linear, or branched hydrocarbon radical with one to 19 carbon atoms bonded to the carbonyl of the ester group).

These copolymers may be cross-linked with the help of agents that may be either of the vinyl type or else of the allyl or methallyl type, such as: tetraallyloxyethane; divinyl benzene; divinyl octane dioate; divinyl dodecane dioate; and divinyl octadecane dioate.

As examples of these copolymers, mention can be made of the following copolymers: vinyl acetate and allyl stearate; vinyl acetate and vinyl laurate; vinyl acetate and vinyl stearate; vinyl acetate and octadecene; vinyl acetate and octadecyl vinyl ether; vinyl propionate and allyl laurate; vinyl propionate and vinyl laurate; vinyl stearate and 1-octadecene; vinyl acetate and 1-dodecene; vinyl stearate and ethyl vinyl ether; vinyl propionate and cetyl vinyl ether; vinyl stearate and allyl acetate; vinyl dimethyl-2,2 octanoate and vinyl laurate; allyl dimethyl-2,2 pentanoate and vinyl laurate; vinyl dimethyl propionate and vinyl stearate; allyl dimethyl propionate and vinyl stearate; vinyl propionate and vinyl stearate, cross-linked with 0.2% divinyl benzene; vinyl dimethyl propionate and vinyl laurate cross-linked with 0.2% divinyl benzene; vinyl acetate and octadecyl vinyl ether, cross-linked with 0.2% tetraallyl oxyethane; vinyl acetate and allyl stearate, cross-linked with 0.2% divinyl benzene; vinyl acetate and 1-octadecene, cross-linked with 0.2% divinyl benzene; and allyl propionate and allyl stearate cross-linked with 0.2% divinyl benzene.

The film-forming polymer may also be selected from silicone resins that are generally soluble or swellable in silicone oils, constituted by cross-linked polyorganosiloxane polymers.

The film-forming polymer may also be present in the composition in the form of particles in dispersion in an aqueous phase or in a non-aqueous solvent phase, generally known as a latex or a pseudolatex. Techniques for preparing such dispersions are well known to the person skilled in the art.

The composition of the invention may comprise a plasticizer agent which encourages the formation of a film with the film-forming polymer. Such a plasticizer agent may be selected from any compound that is known to the person skilled in the art as being suitable for fulfilling the desired function.

Naturally, this list of polymers is not exhaustive.

### Other Coloring Agents

The composition can include one or more diffusing pigments, generating a color by an absorption phenomenon, in a proportion that makes it possible to preserve the interference phenomenon that is responsible for the color of the composition once applied to keratinous materials.

The composition may not comprise other coloring agents than the multilayer interference pigment(s), for example not comprise pigments comprising iron oxides or other pigments producing colors by a phenomenon of absorbing light.

The composition may for example comprise less than 0.5% by weight of pigments producing a color by a phenomenon of absorbing light, for example not more than 0.5% of pigments having iron oxides, for example not more than 0.2%.

When diffusing pigments are present, various diffusing pigments can be envisaged, being selected, for example, from organic lakes or pigments that are selected in particular from the following materials and mixtures thereof:
cochineal carmine;
the organic pigments of azo, anthraquinone, indigo, xanthene, pyrene, quinoline, triphenylmethane, or fluorane dyes; and
organic lakes or insoluble salts of sodium, potassium, calcium, barium, aluminum, zirconium, strontium, titanium, or of acid dyes such as azo, anthraquinone, indigo, xanthene, pyrene, quinoline, triphenylmethane, or fluorine dyes, which dyes may comprise at least one carboxylic or sulfonic acid group.

Organic pigments that may be mentioned include those with the following denominations: D&C Blue No. 4, D&C Brown No. 1, D&C Green No. 5, D&C Green No. 6, D&C Orange No. 4, D&C Orange No. 5, D&C Orange N10, D&C Orange No. 11, D&C Red No. 6, D&C Red No. 7, D&C Red No. 17, D&C Red No. 21, D&C Red No. 22, D&C Red No. 27, D&C Red No. 28, D&C Red No. 30, D&C Red N31, D&C Red No. 33, D&C Red No. 34, D&C Red No. 36, D&C Violet No. 2, D&C Yellow No. 7, D&C Yellow No. 8, D&C Yellow No. 10, D&C Yellow No. 11, FD&C Blue No. 1, FD&C Green No. 3, FD&C Red No. 40, FD&C Yellow No. 5, FD&C Yellow No. 6.

The lake may be supported by an organic support such as colophane or aluminum benzoate, for example.

Particular organic lakes that may be mentioned include those with the following denominations: D&C Red No. 2 Aluminum lake, D&C Red No. 3 Aluminum lake, D&C Red No. 4 Aluminum lake, D&C Red No. 6 Aluminum lake, D&C Red No. 6 Barium lake, D&C Red No. 6 Barium/Strontium lake, D&C Red No. 6 Strontium lake, D&C Red No. 6 Potassium lake, D&C Red No. 7 Aluminum lake, D&C Red No. 7 Barium lake, D&C Red No. 7 Calcium lake, D&C Red No. 7 Calcium/Strontium lake, D&C Red No. 7 Zirconium lake, D&C Red No. 8 Sodium lake, D&C Red No. 9 Aluminum lake, D&C Red No. 9 Barium lake, D&C Red No. 9 Barium/Strontium lake, D&C Red No. 9 Zirconium lake, D&C Red No. 10 Sodium lake, D&C Red No. 19 Aluminum lake, D&C Red No. 19 Barium lake, D&C Red No. 19 Zirconium lake, D&C Red No. 21 Aluminum lake, D&C Red No. 21 Zirconium lake, D&C Red No. 22 Aluminum lake, D&C Red No. 27 Aluminum lake, D&C Red No. 27 Aluminum/Titanium/Zirconium lake, D&C Red No. 27 Barium lake, D&C Red No. 27 Calcium lake, D&C Red No. 27 Zirconium lake, D&C Red No. 28 Aluminum lake, D&C Red No. 30 lake, D&C Red No. 31 Calcium lake, D&C Red No. 33 Aluminum lake, D&C Red No. 34 Calcium lake, D&C Red No. 36 lake, D&C Red No. 40 Aluminum lake, D&C Blue No. 1 Aluminum lake, D&C Green No. 3 Aluminum lake, D&C Orange No. 4 Aluminum lake, D&C Orange No. 5 Aluminum lake, D&C Orange No. 5 Zirconium lake, D&C Orange No. 10 Aluminum lake, D&C Orange No. 17 Barium lake, D&C Yellow No. 5 Aluminum lake, D&C Yellow No. 5 Zirconium lake, D&C Yellow No. 6 Aluminum lake, D&C Yellow No. 7 Zirconium lake, D&C Yellow No. 10 Aluminum lake, FD&C Blue No. 1 Aluminum lake, FD&C Red No. 4 Aluminum lake, FD&C Red No. 40 Aluminum lake, FD&C Yellow No. 5 Aluminum lake, FD&C Yellow No. 6 Aluminum lake.

The chemical materials corresponding to each of the organic coloring substances listed above are mentioned in the work entitled “International Cosmetic Ingredient Dictionary and Handbook”, 1997 edition, pages 371 to 386 and 524 to 528, published by “The Cosmetic, Toiletry, and Fragrance Association”, the contents of which are incorporated herein by reference.

The diffusing pigment can be a composite pigment, comprising a core that is coated at least in part by a husk. In particular, such a composite pigment may be composed of particles comprising an inorganic core and at least one at least partial coating of at least one organic coloring substance. At least one binder may advantageously contribute to fixing the organic coloring substance onto the inorganic core.

The particles of composite pigment may have a variety of forms. In particular, said particles may be in the form of flakes or they may be globular, in particular spherical, and may be hollow or solid. The term “in the form of flakes” means particles for which the ratio of the largest dimension to the thickness is 5 or more. A composite pigment may, for example, have a specific surface area in the range 1 square meter per gram (m²/g) to 1000 m²/g, in particular in the range about 10 m²/g to about 600 m²/g, and in particular in the range about 20 m²/g to about 400 m²/g. The specific surface area is the value measured using the BET (Brunauer-Emmett-Teller) method. The proportion by weight of the core can exceed 50% relative to the total weight of the composite pigment, e.g. lying in the range 50% to 70%, e.g. in the range 60% to 70%.

The coloring agent can also be a colorant.

The colorant can be of plant, animal, or inorganic origin, in particular of plant or inorganic origin, in particular of plant origin. The colorant can be of non-synthetic type.

The colorant can be a natural hydrosoluble or liposoluble colorant.

By way of illustration, suitable natural hydrosoluble coloring agents that may be mentioned in particular for use in the invention are: caramel; beetroot juice; carmine; betanine (beetroot); copper chlorophyllin; methylene blue; anthocyanin (enocianin, black carrot, hibiscus, elder); and riboflavin.

By way of illustration, suitable natural liposoluble coloring agents that may be mentioned in particular for use in the invention are: Sudan red; β-carotene; carotenoids; lycopene; palm oil; Sudan brown; quinoline yellow; xanthophylls (capsanthin, capsorubin, lutein); and curcumin.

Other suitable natural colorants that may be mentioned more particularly are: anthcyanin flowers or fruit or derivatives thereof; flavonoids and tannin extracts of native or fermented plants; juglone; lawsone; extracts of fermented soya, algae, mushrooms, micro-organisms; Flavylium salts non-substituted in position 3 as described in patent EP 1 172 091; extracts of Gesneria Fulgens, Blechum Procerum, Saxifraga; and pigments that can be extracted with an organic or hydro-organic solvent from a culture medium of a microfungus of the monascus Monascus type.

Suitable synthetic colorants that may be mentioned are synthetic liposoluble colorants such as DC Red 17, DC Red 21, DC Red 27, DC Green 6, DC Yellow 11, DC Violet 2, and DC Orange 5, for example.

Suitable synthetic hydrosoluble colorants that may be mentioned are FDC Red 4, DC Red 6, DC Red 22, DC Red 28, DC Red 30, DC Red 33, DC Orange 4, DC Yellow 5, DC Yellow 6, DC Yellow 8, FDC Green 3, DC Green 5, and FDC Blue 1.

### Fillers

The cosmetic composition may include fillers.

The term “fillers” means particles of any form which are insoluble in the composition medium regardless of the temperature at which the composition is manufactured. Said fillers primarily act to modify the rheology or texture of the composition.

Examples of fillers that may be mentioned include talc, mica, silica, kaolin, and powders of polyamide (Nylon® or Orgasol® by Atochem).

The content of fillers is selected in such a manner as not to impede unduly the desired result.

### Active Ingredients and Other Compounds

The cosmetic composition may also contain one or more cosmetically, dermatologically, hygienically, or pharmaceutically active ingredients.

Suitable cosmetically, dermatologically, hygienically, or pharmaceutically active ingredients for use in the compositions of the invention that may be mentioned are moisturizing agents (polyols such as glycerine), vitamins (C, A, E, F, B, or PP), essential fatty acids, essential oils, ceramides, sphingolipids, liposoluble or nanoparticle sun screens, and specific skin treatment active ingredients (protective agents, antibacterials, anti-wrinkle agents, etc). Said active ingredients may be used in concentrations in the range 0.001% to 15%, for example, relative to the total weight of the composition.

The cosmetic composition may also contain ingredients which are routinely used in cosmetics, such as thickeners, surfactants, oligo-elements, moisturizing agents, softeners, sequestrating agents, fragrances, alkalinizing or acidifying agents, preservatives, antioxidants, UV filters, or mixtures thereof.

Depending on the envisaged application, the composition may also include constituents which are conventionally used in the fields under consideration, and which are present in quantities appropriate to the desired dosage or “galenical” form.

### Forms

The cosmetic composition may be in any form that is normally used for topical application, in particular: in anhydrous or non-anhydrous form; in solid form as a loose- or compacted-powder; a casting, e.g. in stick form; or in liquid form as an oily or aqueous solution, an oily or aqueous gel, an oil-in-water emulsion, a water-in-oil emulsion, a multiple emulsion, a dispersion of oil in water due to vesicles located on the oil/water interface, or a spray.

The term “anhydrous composition” means a composition having less than 5% by weight of water, better less than 3%, or even less than 1%. An anhydrous composition need not include water that has been intentionally added during the preparation of the composition.

The composition may in particular be in stick form.

The cosmetic composition may constitute amongst others kinds of makeup: lipstick in liquid or stick form; liquid lip gloss; lipstick paste; blusher; eye-contour composition; eyeliner; mascara; nail varnish; eyeshadow; foundation; and more generally any body or hair makeup.

The composition of the invention may be obtained in accordance with the methods of preparation conventionally used in cosmetics.

### Packaging and Modes of Application

The composition may be packaged in any receptacle or on any support provided for this purpose.

The composition can be applied using an optionally-flocked applicator, e.g. a foam, an endpiece, a paint brush, a felt, a spatula, a sintered element, a brush, a comb, or an optionally-woven fabric.

Application can also be performed using a finger or by placing the composition directly on the substrate that is to be made up, for example by rubbing a stick or by spraying with the help of a piezoelectric device, or by transferring a layer of composition that has previously been deposited on an intermediate substrate.

Where appropriate, the composition can be applied as a base coat that is covered by a top coat for imparting glossiness, for example, or applied as a top coat on a base coat, or even applied between a base coat and a top coat.

The composition can be packaged in a packaging device that makes it possible to see the bulk color of the composition.

The packaging device is a receptacle having a body that is transparent, at least in part, and/or includes a stopper member that is transparent, at least in part.

The composition can be packaged in a device that makes it possible to see both the bulk color of the composition and the color of the composition after application to keratinous materials.

The composition can also be packaged in a device that includes means that are representative of the color that is revealed on application, e.g. a deposit of a layer of the composition or of an ink or a varnish that includes the same coloring agents as the composition.

Packaging the composition can be arranged in such a manner as to make it possible to expose the coloring agent(s) that is/are sensitive to an external stimulus to said stimulus, so as to be able to observe a change in color in the composition within its bulk or after application, while being exposed to the stimulus.

For example, for photochromic coloring agents, the packaging advantageously presents a zone that can optionally be covered, depending on the choice of the consumer.

The invention also provides a composition packaged in this way.

In its packaging, the cosmetic composition can present a zone that can optionally be exposed to light.

### Method of Applying Makeup

The invention also provides a method of making up keratinous material using a composition of the invention.

The method can be for making up the skin, the lips, the nails, the eyelashes, the eyebrows, or the hair.

### Presentation Method and Display Stand

In another of its aspects, the invention also provides a method of presenting a cosmetic composition made in accordance with the invention, the method comprising the steps consisting in:
illuminating at least a first region of a composition sample with diffused lighting so as to make its bulk color appear; and
illuminating at least a second region of a composition sample with directional lighting so as to make its interference color appear.

The first and second regions can be different regions of the same sample of composition.

In a variant, the first and second regions belong to different samples.

Still in a variant, the first and second regions correspond to the same region, and the diffused and directional lighting are applied in alternation.

In another of its aspects, the invention also provides a display stand comprising:
a directional-lighting source;
a diffused-lighting source; and
a support that makes it possible to expose at least one composition sample to the diffused-lighting source, and at least one composition sample to the directional-lighting source.

The display stand can be arranged so as to make it possible to expose the coloring agent(s) that is/are sensitive to an external stimulus to said stimulus, so as to reveal the corresponding variation in color.

In the example of a thermochromic coloring agent, the display stand is arranged so as to expose a composition sample to a heat source so as to cause a change in state, for example.

By way of example, the display stand includes a heated substrate on which the composition is spread in order firstly to reveal the color of the multilayer interference pigment(s) on application, and secondly to induce a change in state in the thermochromic coloring agent.

For a photochromic coloring agent, the lighting used to illuminate the composition in order to reveal the color of the multilayer interference pigment can include UV radiation in order to induce a change in state in the photochromic coloring agent.

### Kit

The present invention also provides a makeup kit comprising:
a first composition of the invention; and
a second composition comprising a cosmetically-acceptable medium for applying under or on top of the first composition.

By way of example, the second composition is for improving the durability of the first composition and/or for modifying the appearance thereof.

### PROPOSED EXAMPLES

The examples 1 to 7 relate to compositions comprising at least one multilayer interference pigment and at least one Xchrome coloring agent.

The quantities given are by weight.

### Example 1

### Lip Gloss

| | |
|---|---|
| Octyl-2 dodecanol | 10 |
| Ditertiobutyl 4-hydroxytoluene | 0.07 |
| Polybutene (monoolefins/isoparaffins 95/5) | 37 |
| (MW: 2060) | |
| A mixture of isopropyl, isobutyl, and n-butyl | 0.6 |
| p-hydroxybenzoates (40/30/30) | |
| Pentaerythrityl tetraisostearate | 11.33 |
| Tridecyl trimellitate | 11 |
| 2-decyl tetradecanoic acid triglyceride | 15 |
| (GUERBET C24) | |
| Multilayer interference pigment* | 12 |
| Xchrome coloring agent** | 3 |

*TIMIRON SILK RED by MERCK.
**REVERSACOL by JAMES ROBINSON.

The effect produced by this lip gloss is a result of the association of a plurality of phenomena. The first relates to the color rendering on application, which is intense and covering. To that is added the Xchromic effect that induces a change in color depending on the type of light and how it is exposed. This lip gloss presents a bulk color that is white.

### Example 2

### Lipstick

| | |
|---|---|
| Tridecyl trimellitate | 11 |
| Liquid lanolin | 10 |
| Isostearyl malate | 12 |
| Acetylated lanolin | 10 |
| Lauric/palmitic/cetylic/stearic acid | 5 |
| triglycerides (50/20/10/10) | |
| Microcrystalline wax (C₂₀-C₆₀) | 3 |
| Protected isopropyl lanolate | 9 |
| Octyl-2-decanol | 15 |
| Phenyl trimethylsiloxytrisiloxane | 4 |
| (VISCOSITY: 20 CST - MW: 372) | |
| Polyethylene wax (MW: 500) | 8 |
| Multilayer interference pigment* | 10 |
| Xchrome coloring agent** | 3 |

*TIMIRON SILK RED by MERCK.
**REVERSACOL by JAMES ROBINSON.

The effect produced by this lipstick is a result of the association of a plurality of phenomena. The first relates to the color rendering on application, which is intense and covering. To that is added the Xchromic effect that induces a change in color depending on the type of light and how it is exposed. This lipstick presents a bulk color that is white.

### Example 3

### Blusher

| | |
|---|---|
| Triethanolamine | 1 |
| Disodium ethylene diamino tetraacetic acid salt, | 0.2 |
| 2H₂0 | |
| Cross-linked carboxyvinyl homopolymer | 0.5 |
| Polyvinylpyrrolidone | 0.6 |
| Glycerol | 5.75 |
| Demineralized water | 77.45 |
| 1,3-butylene glycol | 2 |
| Silica microsphere (3 μm) | 1.5 |
| Multilayer interference pigment* | 8 |
| Xchrome coloring agent** | 3 |

*TIMIRON SILK RED by MERCK.
**Photogenica ® by CATALYST & CHEMICALS.

The effect produced by this blusher is a result of the association of a plurality of phenomena. The first relates to the color rendering on application, which is intense and covering. To that is added the Xchromic effect that induces a change in color depending on the type of light and how it is exposed. This blusher presents a bulk color that is white.

### Example 4

### Water-Based Nail Varnish

| | |
|---|---|
| Tetrasodium pyrophosphate | 0.2 |
| Methoxy-terminated oxyethylene | 0.5 |
| polydimethylsiloxane | |
| A mixture of aliphatic polyurethane, N-methyl | 65 |
| pyrrolidone, triethylamine, and water | |
| (35/8.5/2/54.5) | |
| Glycerol | 1 |
| Demineralized water | 15 |
| Ethyl alcohol (96°) | 2.8 |
| Synthetic laponite (mixed | 1.3 |
| magnesium/lithium/sodium silicate) | |
| Multilayer interference pigment* | 11.2 |
| Xchrome coloring agent** | 3 |

*TIMIRON SILK RED by MERCK.
**Kromafast ® Yellow5GX 02 by KROMACHEM LTD.

The effect produced by this nail varnish is a result of the association of a plurality of phenomena. The first relates to the color rendering on application, which is intense and covering. To that is added the Xchromic effect that induces a change in color depending on the type of light and how it is exposed. This nail varnish presents a bulk color that is white.

### Example 5

### Organic Solvent-Based Nail Varnish

| | |
|---|---|
| Nitrocellulose | 11 |
| N-ethyl o,p-toluenesulfonamide | 5 |
| Alkyde resin | 10 |
| Isopropanol | 4 |
| Multilayer interference pigment* | 10 |
| Xchrome coloring agent** | 3 |
| Butyl acetate/ethyl acetate 50/50 | Qsp |
| | 100 |

*TIMIRON SILK RED by MERCK.
**Kromafast ® Yellow5GX 02 by KROMACHEM LTD.

The effect produced by this nail varnish is a result of the association of a plurality of phenomena. The first relates to the color rendering on application, which is intense and covering. To that is added the Xchromic effect that induces a change in color depending on the type of light and how it is exposed. This nail varnish presents a bulk color that is white.

### Example 6

### Powder Eyeshadow

| | |
|---|---|
| TERTIOBUTYL 4-HYDROXYANISOLE | 0.012 |
| DITERTIOBUTYL 4-HYDROXYTOLUENE | 0.012 |
| WHITE VASELINE | 1.2 |
| OLEIC ALCOHOL | 1.2 |
| PROTECTED LIQUID LANOLIN | 0.66 |
| VASELINE OIL | 6.516 |
| CASTOR OIL | 1.296 |
| ISOPROPYL MYRISTATE | 0.864 |
| PROPYL P-HYDROXYBENZOATE | 0.24 |
| A MIXTURE OF METHYL, ETHYL, PROPYL, BUTYL, | 0.6 |
| ISOBUTYL P-HYDROXY-BENZOATES AND PHENOXY-2 | |
| ETHANOL | |
| Multilayer interference pigment* | 50.3 |
| MAGNESIUM STEARATE | 4 |
| TALC | 30.1 |
| Material having Xchromic properties** | 3 |

*TIMIRON SILK RED by MERCK.
**Photogenica ® by CATALYST & CHEMICALS.

The effect produced by this eyeshadow is a result of the association of a plurality of phenomena. The first relates to the color rendering on application, which is intense and covering. To that is added the Xchromic effect that induces a change in color depending on the type of light and how it is exposed. This eyeshadow presents a bulk color that is white.

### Example 7

### Liquid Eyeshadow

| | |
|---|---|
| DITERTIOBUTYL 4-HYDROXYTOLUENE | 0.09 |
| DISTEARYLDIMETHYLAMMONIUM MODIFIED | 3.74 |
| HECTORITE | |
| LAURIC/PALMITIC/CETYLIC/STEARIC ACID | 6.46 |
| TRIGLYCERIDES (50/20/10/10) | |
| PROPYLENE CARBONATE | 1.22 |
| WHITE BEESWAX | 7.77 |
| PROTECTED SHOREA BUTTER | 1.7 |
| PROTECTED LIQUID FRACTION OF SHEA NUT BUTTER | 0.85 |
| NYLON-12 POWDER | 10.4 |
| ISODODECANE | 35.32 |
| PROPYL P-HYDROXYBENZOATE | 0.17 |
| PROTECTED REFINED PARAFFIN | 3.88 |
| TALC | 10.4 |
| Multilayer interference pigment* | 15 |
| Material having Xchromic properties** | 3 |

*TIMIRON SILK RED by MERCK.
**Photogenica ® by CATALYST & CHEMICALS.

The effect produced by this eyeshadow is a result of the association of a plurality of phenomena. The first relates to the color rendering on application, which is intense and covering. To that is added the Xchromic effect that induces a change in color depending on the type of light and how it is exposed. This eyeshadow presents a bulk color that is white.

The examples 8 to 14 relate to compositions comprising at least one multilayer interference pigment and at least one reflecting pigment with metallic reflection.

### Example 8

### Lip Gloss

| | |
|---|---|
| Octyl-2 dodecanol | 10 |
| DITERTIOBUTYL 4-hydroxytoluene | 0.07 |
| Polybutene (monoolefins/isoparaffins 95/5) | 37 |
| (PM: 2060) | |
| A mixture of isopropyl, isobutyl, and n-butyl | 0.6 |
| p-hydroxybenzoates (40/30/30) | |
| Pentaerythrityl tetraisostearate | 11.33 |
| Tridecyl trimellitate | 11 |
| 2-decyl tetradecanoic acid triglyceride | 15 |
| (GUERBET C24) | |
| Multilayer interference pigment* | 12 |
| Metallic reflection pigment** | 3 |

*TIMIRON SILK BLUE by MERCK.
**METASHINE ME 2040 PS by NIPPON SHEET GLASS.

The effect produced by this lip gloss is a result of the association of a plurality of phenomena. The first relates to the color rendering on application, which is intense and covering. To that are added highlights provided by the particles with metallic reflection. This lipstick presents a bulk color that is white.

### Example 9

### Lipstick

| | |
|---|---|
| Tridecyl trimellitate | 11 |
| Liquid lanolin | 10 |
| Isostearyl malate | 12 |
| Acetylated lanolin | 10 |
| Lauric/palmitic/cetylic/stearic acid | 5 |
| triglycerides (50/20/10/10) | |
| Microcrystalline wax (C20-C60) | 3 |
| Protected isopropyl lanolate | 9 |
| Octyl-2-decanol | 15 |
| Phenyl trimethylsiloxytrisiloxane | 4 |
| (VISCOSITY: 20 CST-PM: 372) | |
| Polyethylene wax (PM: 500) | 8 |
| Multilayer interference pigment* | 10 |
| Metallic reflection pigment** | 3 |

*TIMIRON SILK RED by MERCK.
**METASHINE ME 2040 PS by NIPPON SHEET GLASS.

The effect produced by this lipstick is a result of the association of a plurality of phenomena. The first relates to the color rendering on application, which is intense and covering. To that are added highlights provided by the particles with metallic reflection. This lipstick presents a bulk color that is white.

### Example 10

### Blusher

| | |
|---|---|
| Triethanolamine | 1 |
| Disodium ethylene diamino tetraacetic acid salt, | 0.2 |
| 2H₂0 | |
| Cross-linked carboxyvinyl homopolymer | 0.5 |
| Polyvinylpyrrolidone | 0.6 |
| Glycerol | 5.75 |
| Demineralized water | 77.45 |
| 1,3-butylene glycol 1,3 | 2 |
| Silica microsphere (3 μm) | 1.5 |
| Multilayer interference pigment* | 8 |
| Metallic reflection pigment** | 3 |

*TIMIRON SILK RED by MERCK.
**METASHINE ME 2040 PS by NIPPON SHEET GLASS.

The effect produced by this blusher is a result of the association of a plurality of phenomena. The first relates to the color rendering on application, which is intense and covering. To that are added highlights provided by the particles with metallic reflection. This blusher presents a bulk color that is white.

### Example 11

### Water-Based Nail Varnish

| | |
|---|---|
| Tetrasodium pyrophosphate | 0.2 |
| Methoxy-terminated oxyethylene | 0.5 |
| polydimethylsiloxane | |
| A mixture of aliphatic polyurethane, N-methyl | 65 |
| pyrrolidone, triethylamine, and water | |
| (35/8.5/2/54.5) | |
| Glycerol | 1 |
| Demineralized water | 15 |
| Ethyl alcohol (96°) | 2.8 |
| Synthetic laponite (mixed | 1.3 |
| magnesium/lithium/sodium silicate) | |
| Multilayer interference pigment* | 11.2 |
| Metallic reflection pigment** | 3 |

*TIMIRON SILK RED by MERCK.
**METASHINE ME 2040 PS by NIPPON SHEET GLASS.

### Example 12

### Nail Varnish Based on Organic Solvent

| | |
|---|---|
| Nitrocellulose | 11 |
| N-ethyl o,p-toluenesulfonamide | 5 |
| Alkyde resin | 10 |
| Isopropanol | 4 |
| Multilayer interference pigment* | 10 |
| Metallic reflection pigment** | 3 |
| Butyl acetate/ethyl acetate 50/50 | Qsp |
| | 100 |

*XIRONA SILVER by MERCK.
**METASHINE ME 2040 PS by NIPPON SHEET GLASS.

The particles with metallic reflection provide highlights. This nail varnish presents a bulk color that is white.

### Example 13

### Powder Eyeshadow

| | |
|---|---|
| TERTIOBUTYL 4-HYDROXYANISOLE | 0.012 |
| DITERTIOBUTYL 4-HYDROXYTOLUENE | 0.012 |
| WHITE VASELINE | 1.2 |
| OLEIC ALCOHOL | 1.2 |
| PROTECTED LIQUID LANOLIN | 0.66 |
| VASELINE OIL | 6.516 |
| CASTOR OIL | 1.296 |
| ISOPROPYL MYRISTATE | 0.864 |
| PROPYL P-HYDROXYBENZOATE | 0.24 |
| A MIXTURE OF METHYL, ETHYL, PROPYL, BUTYL, | 0.6 |
| ISOBUTYL P-HYDROXY-BENZOATES AND PHENOXY-2 | |
| ETHANOL | |
| Multilayer interference pigment* | 50.3 |
| MAGNESIUM STEARATE | 4 |
| TALC | 30.1 |
| Metallic reflection pigment** | 3 |

*TIMIRON SILK RED by MERCK.
**METASHINE ME 2040 PS by NIPPON SHEET GLASS.

### Example 14

### Liquid Eyeshadow

| | |
|---|---|
| DITERTIOBUTYL 4-HYDROXYTOLUENE | 0.09 |
| DISTEARYLDIMETHYLAMMONIUM MODIFIED | 3.74 |
| HECTORITE | |
| LAURIC/PALMITIC/CETYLIC/STEARIC ACID | 6.46 |
| TRIGLYCERIDES (50/20/10/10) | |
| PROPYLENE CARBONATE | 1.22 |
| WHITE BEESWAX. | 7.77 |
| PROTECTED SHOREA BUTTER | 1.7 |
| PROTECTED LIQUID FRACTION OF SHEA NUT BUTTER | 0.85 |
| NYLON-12 POWDER | 10.4 |
| ISODODECANE | 35.32 |
| PROPYL P-HYDROXYBENZOATE | 0.17 |
| PROTECTED REFINED PARAFFIN | 3.88 |
| TALC | 10.4 |
| Multilayer interference pigment* | 15 |
| Metallic reflection pigment** | 3 |

*TIMIRON SILK RED by MERCK.
**METASHINE ME 2040 PS by NIPPON SHEET GLASS.

The background is preferably spread out well in order to reveal the color effect. A flocked endpiece can be used for this purpose.

The examples 15 to 20 relate to compositions having at least one multilayer interference pigment and at least one diffracting pigment.

### Example 15

### Lip Gloss

| | |
|---|---|
| Octyl-2 dodecanol | 10 |
| DITERTIOBUTYL 4-hydroxytoluene | 0.07 |
| Polybutene (monoolefins/isoparaffins 95/5) | 37 |
| (PM: 2060) | |
| A mixture of isopropyl, isobutyl, and n-butyl | 0.6 |
| p-hydroxybenzoates (40/30/30) | |
| Pentaerythrityl tetraisostearate | 11.33 |
| Tridecyl trimellitate | 11 |
| 2-decyl tetradecanoic acid triglyceride | 15 |
| (GUERBET C24) | |
| Multilayer interference pigment* | 12 |
| Diffracting pigment** | 3 |

*TIMIRON SILK RED by MERCK.
**Diffracting pigment SPECTRAFLAIR by FLEX PRODUCTS

The effect produced by this lip gloss is a result of the association of a plurality of phenomena. The first relates to the color rendering on application, which is intense and covering. To that is added the “rainbow effect” provided by the diffracting pigment. This lip gloss presents a bulk color that is white.

### Example 16

### Lipstick

| | |
|---|---|
| Tridecyl trimellitate | 11 |
| Liquid lanolin | 10 |
| Isostearyl malate | 12 |
| Acetylated lanolin | 10 |
| Lauric/palmitic/cetylic/stearic acid | 5 |
| triglycerides (50/20/10/10) | |
| Microcrystalline wax (C20-C60) | 3 |
| Protected isopropyl lanolate | 9 |
| Octyl-2-decanol | 15 |
| Phenyl trimethylsiloxytrisiloxane | 4 |
| (VISCOSITY: 20 CST-PM: 372) | |
| Polyethylene wax (PM: 500) | 8 |
| Multilayer interference pigment* | 10 |
| Diffracting pigment** | 3 |

*TIMIRON SILK RED by MERCK.
**Diffracting pigment SPECTRAFLAIR by FLEX PRODUCTS

The effect produced by this lipstick is a result of the association of a plurality of phenomena. The first relates to the color rendering on application, which is intense and covering. To that is added the “rainbow effect” provided by the diffracting pigment. This lipstick presents a bulk color that is white.

### Example 17

### Blusher

| | |
|---|---|
| Triethanolamine | 1 |
| Disodium ethylene diamino tetraacetic acid salt, | 0.2 |
| 2H₂0 | |
| Cross-linked carboxyvinyl homopolymer | 0.5 |
| Polyvinylpyrrolidone | 0.6 |
| Glycerol | 5.75 |
| Demineralized water | 77.45 |
| 1,3-butylene glycol 1,3 | 2 |
| Silica microsphere (3 μm) | 1.5 |
| Multilayer interference pigment* | 8 |
| Diffracting pigment** | 3 |

*TIMIRON SILK RED by MERCK.
**Diffracting pigment SPECTRAFLAIR by FLEX PRODUCTS

The effect produced by this blusher is a result of the association of a plurality of phenomena. The first relates to the color rendering on application, which is intense and covering. To that is added the “rainbow effect” provided by the diffracting pigment. This blusher presents a bulk color that is white.

### Example 18

### Water-Based Nail Varnish

| | |
|---|---|
| Tetrasodium pyrophosphate | 0.2 |
| Methoxy-terminated oxyethylene | 0.5 |
| polydimethylsiloxane | |
| A mixture of aliphatic polyurethane, N-methyl | 65 |
| pyrrolidone, triethylamine, and water | |
| (35/8.5/2/54.5) | |
| Glycerol | 1 |
| Demineralized water | 15 |
| Ethyl alcohol (96°) | 2.8 |
| Synthetic laponite (mixed | 1.3 |
| magnesium/lithium/sodium silicate) | |
| Multilayer interference pigment* | 11.2 |
| Diffracting pigment** | 3 |

*TIMIRON SILK BLUE by MERCK.
**Diffracting pigment SPECTRAFLAIR by FLEX PRODUCTS

The effect produced by this nail varnish is a result of the association of a plurality of phenomena. The first relates to the color rendering on application, which is intense and covering. To that is added the “rainbow effect” provided by the diffracting pigment. This nail varnish presents a bulk color that is white.

### Example 19

### Anhydrous Nail Varnish

| | |
|---|---|
| Nitrocellulose | 11 |
| N-ethyl o,p-toluenesulfonamide | 5 |
| Alkycle resin | 10 |
| Isopropanol | 4 |
| Multilayer interference pigment* | 10 |
| Diffracting pigment** | 3 |
| Butyl acetate/ethyl acetate 50/50 | Qsp 100 |

*XIRONA SILVER by MERCK.
**Diffracting pigment SPECTRAFLAIR by FLEX PRODUCTS

The effect produced by this nail varnish is a result of the association of a plurality of phenomena. The first relates to the color rendering on application, which is intense and covering. To that is added the “rainbow effect” provided by the diffracting pigment. This nail varnish presents a bulk color that is white.

### Example 20

### Powder Eyeshadow

| | |
|---|---|
| TERTIOBUTYL 4-HYDROXYANISOLE | 0.012 |
| DITERTIOBUTYL 4-HYDROXYTOLUENE | 0.012 |
| WHITE VASELINE | 1.2 |
| OLEIC ALCOHOL | 1.2 |
| PROTECTED LIQUID LANOLIN | 0.66 |
| VASELINE OIL | 6.516 |
| CASTOR OIL | 1.296 |
| ISOPROPYL MYRISTATE | 0.864 |
| PROPYL P-HYDROXYBENZOATE | 0.24 |
| A MIXTURE OF METHYL, ETHYL, PROPYL, BUTYL, | 0.6 |
| ISOBUTYL P-HYDROXY-BENZOATES AND PHENOXY-2 | |
| ETHANOL | |
| Multilayer interference pigment* | 50.3 |
| MAGNESIUM STEARATE | 4 |
| TALC | 30.1 |
| Diffracting pigment** | 3 |

*TIMIRON SILK RED by MERCK.
**Diffracting pigment SPECTRAFLAIR by FLEX PRODUCTS

The effect produced by this powder eyeshadow is a result of the association of a plurality of phenomena. The first relates to the color rendering on application, which is intense and covering. To that is added the “rainbow effect” provided by the diffracting pigment. This powder eyeshadow presents a bulk color that is white.

### Example 21

### Liquid Eyeshadow

| | |
|---|---|
| DITERTIOBUTYL 4-HYDROXYTOLUENE | 0.09 |
| DISTEARYLDIMETHYLAMMONIUM MODIFIED | 3.74 |
| HECTORITE | |
| LAURIC/PALMITIC/CETYLIC/STEARIC ACID | 6.46 |
| TRIGLYCERIDES (50/20/10/10) | |
| PROPYLENE CARBONATE | 1.22 |
| WHITE BEESWAX. | 7.77 |
| PROTECTED SHOREA BUTTER | 1.7 |
| PROTECTED LIQUID FRACTION OF SHEA NUT BUTTER | 0.85 |
| NYLON-12 POWDER | 10.4 |
| ISODODECANE | 32.32 |
| PROPYL P-HYDROXYBENZOATE | 0.17 |
| PROTECTED REFINED PARAFFIN | 3.88 |
| TALC | 10.4 |
| Multilayer interference pigment* | 15 |
| Diffracting pigment** | 3 |

*TIMIRON SILK BLUE by MERCK.
**Diffracting pigment SPECTRAFLAIR by FLEX PRODUCTS

The effect produced by this eyeshadow is a result of the association of a plurality of phenomena. The first relates to the color rendering on application, which is intense and covering. To that is added the “rainbow effect” provided by the diffracting pigment. This eyeshadow presents a bulk color that is white.

### Set for a Two-Coatings Application

The composition according to the invention can be associated to a top coat or a base coat. Examples 22 and 23 providing associations are given hereinafter. In examples 22 and 23, one of the first and second composition may be replaced by one of the compositions of examples 1 to 21.

### Example 22

### Lipstick

First composition

| | |
|---|---|
| Tridecyl trimellitate | 11 |
| Liquid lanolin | 10 |
| Isostearyl malate | 13 |
| Acetylated lanolin | 10 |
| Lauric/palmitic/cetylic/stearic acid | 5 |
| triglycerides (50/20/10/10) | |
| Microcrystalline wax (C20-C60) | 3 |
| Protected isopropyl lanolate | 10 |
| Octyl-2-decanol | 16 |
| Phenyl trimethylsiloxytrisiloxane | 4 |
| (VISCOSITY: 20 CST-PM: 372) | |
| Polyethylene wax (PM: 500) | 8 |
| Multilayer interference pigment* | 10 |

*TIMIRON SILK RED by MERCK.

Second composition (top coat)

| | |
|---|---|
| Octyl-2 dodecanol | 10 |
| DITERTIOBUTYL 4-hydroxytoluene | 0.07 |
| Polybutene (monoolefins/isoparaffins 95/5) | 50 |
| (PM: 2060) | |
| A mixture of isopropyl, isobutyl, and n-butyl | 0.4 |
| p-hydroxybenzoates (40/30/30) | |
| Pentaerythrityl tetraisostearate | 11.33 |
| Tridecyl trimellitate | 12 |
| 2-decyl tetradecanoic acid triglyceride | 11 |
| (GUERBET C24) | |
| Effect pigment** | 3 |

**METASHINE ME 2040 PS by NIPPON SHEET GLASS.

The first composition makes it possible to generate a uniform layer of makeup having a red color that is very intense. The second composition serves to create a magnifying glass effect accompanied by a silver highlight that gives relief to the overall result of the makeup.

### Example 23

### Lipstick

Base coat

| | |
|---|---|
| Tridecyl trimellitate | 12 |
| Liquid lanolin | 11 |
| Isostearyl malate | 14 |
| Acetylated lanolin | 11 |
| Lauric/palmitic/cetylic/stearic acid | 6 |
| triglycerides (50/20/10/10) | |
| Microcrystalline wax (C20-C60) | 3 |
| Protected isopropyl lanolate | 10 |
| Octyl-2-decanol | 16 |
| Phenyl trimethylsiloxytrisiloxane | 4 |
| (VISCOSITY: 20 CST-PM: 372) | |
| Polyethylene wax (PM: 500) | 8 |
| Black iron oxide (Fe₃O₄) pigment* | 5 |

*TIMIRON SILK RED by MERCK.

Composition containing multilayer interference pigment

| | |
|---|---|
| Octyl-2 dodecanol | 10 |
| DITERTIOBUTYL 4-hydroxytoluene | 0.07 |
| Polybutene (monoolefins/isoparaffins 95/5) | 45.2 |
| (PM: 2060) | |
| A mixture of isopropyl, isobutyl, and n-butyl | 0.4 |
| p-hydroxybenzoates (40/30/30) | |
| Pentaerythrityl tetraisostearate | 11.33 |
| Tridecyl trimellitate | 12 |
| 2-decyl tetradecanoic acid triglyceride | 11 |
| (GUERBET C24) | |
| Multilayer interference pigment** | 10 |

**TIMIRON SILK RED by MERCK.

The base coat is applied first and serves to generate a uniform layer of makeup having a very intense black color. The composition containing the multilayer interference pigment is applied thereon and serves to create a color effect giving a very intense makeup result.

The following description relates to another embodiment of the invention.

### Solid Cosmetic

### Measuring the Coverage of the Cosmetic

The compositions of the cosmetic are melted and mixed together, and then the coverage is measured as described above for a single composition.

### Pasty Compound

The medium may include a pasty compound selected for example from:
polymer or non-polymer silicone compounds;
polymer or non-polymer fluorinated compounds;
vinyl polymers, and in particular:
   homopolymers of olefins;
   copolymers of olefins;
   homopolymers and copolymers of hydrogenated dienes;
   homo- or copolymer linear or branched oligomers of alkyl(meth)acrylates preferably having a C₈-C₃₀ alkyl group;
   homo- and copolymer oligomers of vinyl esters having C₈-C₃₀ alkyl groups;
   homo- and copolymer oligomers of vinyl ethers having C₈-C₃₀ alkyl groups;
liposoluble polyethers resulting from polyetherification between one or more C₂-C₁₀₀ diols, preferably C₂-C₅₀ diols;
esters; and
mixtures thereof.

The pasty compound is preferably a polymer, in particular a hydrocarbon.

Silicone and/or Fluorinated Pasty Compounds

An example of a silicone and fluorinated pasty compound is polymethyl trifluoropropyl methylaklyl dimethylsiloxane manufactured under the name X22-1088 by the supplier SHIN-ETSU.

When the pasty compound is a silicone and/or fluorinated polymer, the composition advantageously includes a compatibility agent such as short-chain esters such as isodecyl nepentanoate.

### Polyether Pasty Compounds

Amongst liposoluble polyethers, particular preference is given to copolymers of ethylene-oxide and/or propylene-oxide with C₆-C₃₀ long-chain alkylene-oxides, more preferably such that the ratio by weight of ethylene-oxide and/or propylene-oxide relative to alkylene-oxides in the copolymer lies in the range 5:95 to 70:30. In this family, particular mention is made of the copolymers in which the long chain alkylene-oxides are disposed in blocks having mean molecular weight lying in the range 1000 to 10,000, e.g. a block copolymer of polyoxyethylene and polydodecyl glycol such as the dodecanediol ethers (22 mol) and polyethylene glycol (45 OE) sold under the trademark ELFACOS ST9 by Akzo Nobel.

Amongst these esters, the following are particularly preferred:
esters of an oligomer glycerol, in particular esters of diglycerol, in particular condensates of adipic acid and glycerol, for which some of the hydroxyl groups of the glycerol have reacted with a mixture of fatty acids such as stearic acid, capric acid, stearic acid and isostearic acid, and 12-hydroxystearic acid, in particular like those sold under the trademark Softisan 649 by the supplier Sasol;
arachidyl propionate sold under the trademark Waxenol 801 by Alzo;
phytosterol esters;
the non-cross-linked polyesters that result from polycondensation between a diacid or a C₄-C₅₀ linear or branched carboxylic polyacid and a C₂-C₅₀ diol or polyol;
the aliphatic ester esters resulting from esterification of an aliphatic hydroxycarboxylic acid ester by an aliphatic carboxylic acid; and mixtures thereof.

The aliphatic carboxylic acid comprises 4 to 30 and preferably 8 to 30 carbon atoms. It is preferably selected from hexanoic acid; heptanoic acid; octanoic acid; ethyl-2 hexanoic acid; nonanoic acid; decanoic acid; undecanoic acid; dodecanoic acid; tridecanoic acid; tetradecanoic acid; pentadecanoic acid; hexadecanoic acid; hexyldecanoic acid; heptadecanoic acid: octadecanoic acid; isostearic acid; nonadecanoic acid; eicosanoic acid; isoarachidic acid; octyldodecanoic acid; heneicosanoic acid; docosanoic acid; and mixtures thereof.

The aliphatic carboxylic acid is preferably branched.

The hydroxy aliphatic carboxylic acid ester is advantageously derived from a hydroxylated aliphatic carboxylic acid having 2 to 40 carbon atoms, preferably 10 to 34 carbon atoms, and better 12 to 28 carbon atoms, and 1 to 20 hydroxyl groups, preferably 1 to 10 hydroxyl groups and better 1 to 6 hydroxyl groups. The hydroxy aliphatic carboxylic acid ester is selected from:

a) partial or total esters of saturated, linear monohydroxylated aliphatic carboxylic monoacids;

b) partial or total esters of unsaturated monohydroxylated aliphatic carboxylic monoacids;

c) partial or total esters of saturated monohydroxylated aliphatic carboxylic polyacids;

d) partial or total esters of saturated polyhydroxylated aliphatic carboxylic polyacids;

e) partial or total esters of C₂-C₁₆ aliphatic polyols that have reacted with mono- or polyhydroxylated aliphatic carboxylic mono- or polyacids; and

mixtures thereof.

The aliphatic ester esters are advantageously selected from:
the ester that results from the esterification reaction of hydrogenated castor oil with isostearic acid in proportions 1 to 1 (1/1) or hydrogenated castor oil monoisostearate;
the esters that result from the esterification reaction of hydrogenated castor oil with isostearic acid in proportions of 1 to 2 (1/2) or hydrogenated castor oil diisostearate;
the ester resulting from the esterification reaction of hydrogenated castor oil with isostearic acid in the proportions of 1 to 3 (1/3) or hydrogenated castor oil trisostearate; and
mixtures thereof.

Amongst the pasty compounds of vegetable origin, it is preferable to select a mixture of soy bean sterols and oxypropylene (5 OP) oxyethylene (5 OE) pentaerythritol sold under the reference Lanolide by the supplier VEVY.

The pasty compounds preferably represent 1% to 99%, preferably 1% to 60%, better 2% to 30%, and better still 5% to 15% by weight of each composition of the solid cosmetic.

In particular, the pasty compound is an ester as defined above suitable for being associated in the cosmetic composition in a ratio by weight between the pasty compound and the ester lying in the range 0.25 to 0.75, in particular 0.3 to 0.6.

The cosmetic composition of the invention may present a continuous fatty phase, possibly containing less than 5% water, and in particular less than 1% water relative to its total weight.

### Fatty Phase

Each composition, in particular when the cosmetic is for application to the lips, may include at least one fatty phase and in particular at least one fat that is liquid at ambient temperature (25° C.) and at atmospheric pressure and/or one fat that is solid at ambient temperature and at atmospheric pressure such as waxes, gums, and mixtures thereof.

The fatty phase may also contain agents for gelling and structuring oils of organic nature and/or lipophilic organic solvents.

As a liquid fat, the fatty phase of the composition of the invention may comprise at least one volatile or non-volatile oil, or a mixture thereof.

The term “volatile oil” is used in the invention to mean any oil liable to evaporate on being in contact with the skin for less than 1 hour, at ambient temperature, and at atmospheric pressure. Volatile oils of the invention are cosmetic volatile oils, that are liquid at ambient temperature, having non-zero vapor pressure at ambient temperature and atmospheric pressure, in particular lying in the range 1.01 millimeters of mercury (mmHg) to 300 mmHg (i.e. 1.33 pascals (Pa) to 40,000 Pa), and preferably being greater than 0.3 mmHg (30 Pa).

The term “non-volatile oil” is used to mean an oil that remains on the skin at ambient temperature and at atmospheric pressure for at least several hours and in particular having a vapor pressure of less than 0.01 mmHg (1.33 Pa).

These volatile or non-volatile oils may be hydrocarbon oils in particular of vegetable origin, silicone oils, or mixtures thereof. The term “hydrocarbon oil” is used to mean an oil containing mainly atoms of hydrogen and of carbon, possibly together with atoms of oxygen, nitrogen, sulfur, and/or phosphorus.

The volatile hydrocarbon coils can be selected from hydrocarbon oils having 8 to 16 carbon atoms, in particular C₈-C₁₆ branched alkanes such as C₈-C₁₆ isoalkanes of petroleum origin (also known as isoparaffins), such as isododecane (also known as 2,2,4,4,6-pentamethylheptane), isodecane, isohexadecane, and for example the oils sold under the trade names Isopars® or Permethyls®, C₈-C₁₆ branched esters such as isohexyl neopentanoate, and mixtures thereof. Other volatile hydrocarbon oils such as petroleum distillates, in particular those sold under the name Shell Solt® by the supplier Shell, can also be used.

As volatile oils, it is also possible to use volatile silicones, such as for example volatile linear or cyclic silicone oils, in particular those having viscosity≦8 centistokes (8×10⁻⁶ m²/s), and having in particular 2 to 7 silicon atoms, these silicones optionally including alkyl or alkoxy groups having 1 to 10 carbon atoms. As a volatile silicone oil suitable for use in the invention, mention can be made in particular of octamethyl cyclotetrasiloxane, decamethyl cyclotetrasiloxane, dodecamethyl cyclohexasiloxane, heptamethyl hexyltrisiloxane, heptamethyloctyl trisiloxane, hexamethyl disiloxane, octamethyl trisiloxane, decamethyl tetrasiloxane, dodecamethyl pentasiloxane, and mixtures thereof.

The volatile oil may be present in the composition of the invention at a concentration lying in the range 0.1% to 98% by weight, in particular 1% to 65% by weight, and more particularly 2% to 50% by weight relative to the total weight of the composition.

The non-volatile oils may be selected in particular from hydrocarbon oils that are fluorinated, where appropriate, and/or non-volatile silicone oils.

As non-volatile hydrocarbon oils, mention can be made in particular of the following:
hydrocarbon oils of animal origin;
hydrocarbon oils of vegetable origin such as triglycerides constituted by fatty acid esters and glycerol in which the fatty acids can have a variety of chain lengths in the range C₈ to C₂₄, said chains possibly being linear or branched, saturated or unsaturated; these oils are in particular the following oils: wheat germ, sunflower, grape seed, sesame, maize, apricot, castor, karite, avocado, olive, soy bean, sweet almond, palm, colza, cotton, hazelnut, macadamia, jojoba, alfalfa, poppy, Hokkaito squash, sesame, vegetable marrow, colza, black current, evening primrose, millet, barley, quinoa, rye, safflower, candlenut, passion fruit, muscat rose tree; karite butter; or indeed triglycerides of caprylic and capric acids such as those sold by the supplier Stearineries Dubois or those sold under the names Miglyol 810®, 812®, and 818® by the supplier Dynamic Nobel:
synthetic ethers having 10 to 40 carbon atoms;
linear or branched hydrocarbons of mineral or synthetic origin such as Vaseline, polydecanes, hydrogen polyisobutene such as parleam, squalene, and mixtures thereof;
synthetic esters such as oils having the formula R₁COOR₂ in which R₁ represents the residue of a linear or branches fatty acid having 1 to 40 carbon atoms and R₂ represents a hydrocarbon chain, in particular a branch chain containing 1 to 40 carbon atoms on the condition that R₁+R₂ is greater than or equal to 10, for example Purcellin oil (cetostearyl octanoate), isopropyl myristate, isopropyl palmitate, benzoates of C₁₂ to C₁₅ alcohols, hexyl laurate, diisopropyl adipate, isononyl isononanoate, 2-ethyl-hexyl palmitate, isostearyl isostearate, heptanoates, octanoates, decanoates, or ricinoleates of alcohols or of polyalcohols such as propylene glycol dioctanoate; hydroxyl esters such as isostearyl lactate, di-isostearyl malate; polyol esters, and pentaerythritol esters;
fatty alcohols that are liquid at ambient temperature having a branched and/or unsaturated carbon chain with 12 to 26 carbon atoms such as octyl dodecanol, isotearylic alcohol, oleic alcohol, 2-hexyldecanol, 2-butyloctanol, and 2-undecylpentadecanol; and
higher fatty acids such as oleic acid, linoleic acid, linolenic acid, and mixtures thereof.

The non-volatile silicone oils usable in a composition of a cosmetic of the invention may be non-volatile polydimethylsiloxanes (PDSM), polydimethylsiloxanes including alkyl or alcoxy groupings that are pendant and/or at the ends of the silicone chain, groupings each having 2 to 24 carbon atoms, phenyl silicones such as phenyl trimethicones, phenyl dimethicones, phenyl trimethylsiloxy diphenylsiloxanes, diphenyl dimethicones, diphenyl methyldiphenyl trisiloxanes, and 2-phenylethyl trimethylsiloxysilicates, and mixtures thereof.

The non-volatile oils may be present in a composition of a cosmetic of the invention at a concentration lying in the range 0.01% to 90% by weight, in particular 0.1% to 85% by weight, and more particularly 1% to 70% by weight, relative to the total weight of the composition.

The oils may represent 0% to 99% of the total weight of each composition, preferably 0.05% to 60%, better 1% to 35%.

The oils may have molecular weight lying in the range 650 grams per mole (g/mol) to 10,000 g/mol, preferably in the range 750 g/mol to 7500 g/mol.

In an embodiment, each composition has an oily phase comprising at least 70% by weight of an oil having molar weight lying in the range 650 g/mol to 10,000 g/mol, preferably in the range 750 g/mol to 7500 g/mol. Advantageously, the oily phase has more than 80%, preferably more than 85% by weight of an oil of molecular weight lying in the range 650 g/mol to 10,000 g/mol, preferably 750 g/mol to 7500 g/mol.

The oil of high molecular weight may be selected from lipophilic polymers:
the esters of linear fatty acids with a total number of carbon atoms lying in the range 35 to 70;
hydroxylated esters;
aromatic esters;
fatty alcohol esters or C₂₄-C₂₈ branched fatty acids;
silicone oils;
oils of vegetable origin; and
mixtures thereof.

The oil of high molecular weight may be selected from polybutylenes; hydrogenated polyisobutylenes; polydecenes; hydrogenated polydecenes; copolymers of vinyl pyrrolidone (PVP) such as PVP and hexadecene copolymer; pentaerythrityl tetrapelargonate; 2-polyglycerol triisostearate; tridecyl trimellitate; triisoarachidyl citrate; pentaerythrityl tetraisononanoate; glyceryl triisostearate; pentaerythrityl tetraisostearate; glyceryl tri 2-decyl tetradecanoate; pentaerythrityl tetra 2-decyl tetradecanoate; phenyl silicones; sesame oil; and mixtures thereof.

Each composition may also include a fatty body that is solid at ambient temperature and at atmospheric pressure, e.g. selected from waxes, gums, and mixtures thereof. The solid fatty body may represent 0.01% to 50% and in particular 0.1% to 40%, and more particularly 0.2% to 30% by weight relative to the total weight of the fatty phase.

### Waxes and Gums

Each composition may contain at least one wax.

The term “wax” is used to mean a lipophilic fatty compound that is solid at ambient temperature (25° C.), with a reversible solid/liquid change of state, presenting a melting temperature greater than 30° C. and possibly as high as 200° C., of hardness greater than 0.5 MPa, and presenting in the solid state an anisotropic crystal organization. By raising the wax to its melting temperature, it is possible to make it miscible with oils and to form a mixture that is microscopically uniform, but on bringing the temperature of the mixture back to ambient temperature, the wax is caused to recrystallize in the oils of the mixture.

Waxes suitable for structuring the composition in particular in the form of a stick, are compounds that are solid at ambient temperature; they may be hydrocarbon, fluorinated, and/or silicone waxes, and they may be of vegetable, mineral, animal, and/or synthetic origin. In particular, they present a melting temperature higher than 40° C., better higher than 45° C.

Waxes that can be used include in particular those generally used in the field of cosmetics: they are in particular of natural origin such as beeswax; Carnauba wax; Candelilla wax; Ouricoury wax; Japan wax; cork fiber or sugar cane wax; rice wax; Montan wax; paraffin; lignite or microcrystalline waxes; cerezin or ozokerite; hydrogenated oils such as jojoba oil; synthetic waxes such as polyethylene waxes obtained by polymerizing or copolymerizing ethylene and Fischer-Tropsch waxes; or fatty acid esters such as octacosanyl stearate; glycerides concrete at 40° C. and better at 45° C.; silicone waxes such as alkyl- or alkoxydimethicones with an alkyl or an alkoxy chain having 10 to 45 carbon atoms; esters of poly(di)methylsiloxane that are solid at 40° C., in which the ester chain has at least 10 carbon atoms; and mixtures thereof.

The gums usable in the invention are generally presented in oil-solubilized form, the polymers are solid at ambient temperature and the resins may be liquid or solid at ambient temperature.

The term “gum” is used to designate a fatty body that is in the form of a polymer that is solid at ambient temperature, having a mass average molecular weight of 50,000 to 1,000,000. Gum is often sold as a dispersion in an organic solvent, of silicone oil type.

The nature and the quantity of gums or waxes are functions of the textures and mechanical properties that are desired. By way of indication, the wax may represent 0.01% to 50%, preferably 2% to 40%, better 5% to 30% of the total weight of each composition of the cosmetic.

In particular, the wax may be present in the form of a wax-in-water emulsion.

The wax may be present in each composition in an amount lying in the range 0.01% to 50% by weight, in particular 0.1% to 30% by weight, and more specifically 0.2% to 20% by weight, relative to the total weight of the composition.

### Preparation

Each composition may be obtained using the methods of preparation that are conventionally used in cosmetics or in dermatology.

The cosmetic may be extruded, compacted, or cast to form a stick or into a cup. The compositions of the cosmetic may be coextruded.

Amongst other kinds of makeup, the cosmetic may constitute a cosmetic for application to the lips, e.g. a lip gloss, a lipstick, a lip balm, a blusher, a foundation, an eyeliner, an eyeshadow, a makeup base, a cosmetic for making up the body or the hair.

### Packaging and Modes of Application

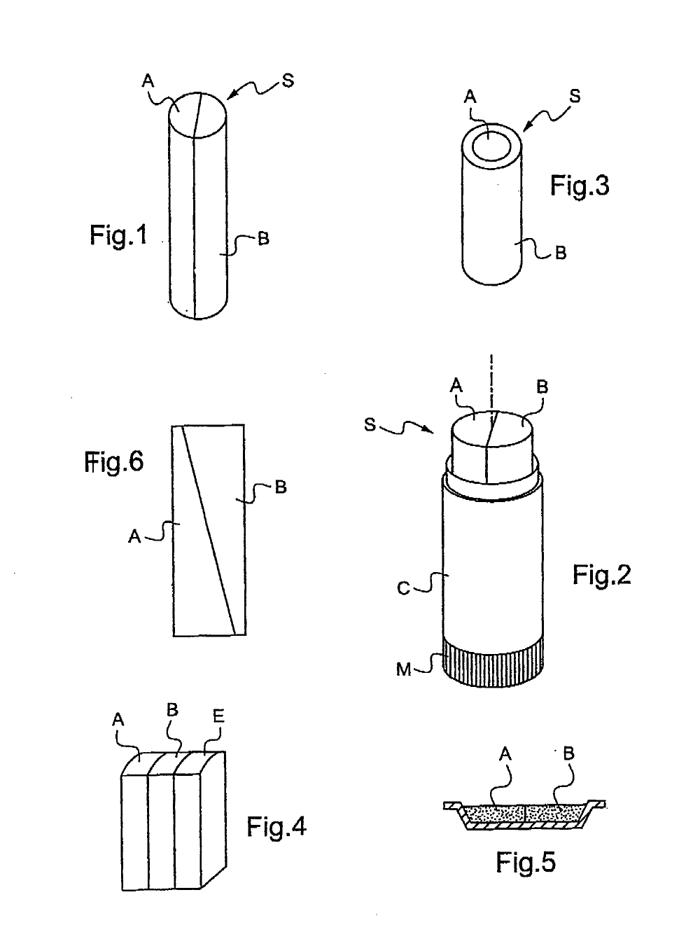

By way of example, FIG. 1 shows a stick S having two blocks A and B extending over the entire length of the stick.

Each block A or B occupies, for example, half of the stick, and the two blocks meet on a diameter of the stick, for example.

The stick S may be packaged in a packaging, and dispenser device comprising for example a body C in which the stick is received at least in part, as shown in FIG. 2, and a drive mechanism M serving to move the stick relative to the body C.

The packaging and dispenser device may be of any type adapted to packaging a stick and a drive mechanism M and for example it includes, at its bottom end, a knob that can be turned.

The two blocks A and B may optionally be equal proportions within the stick.

One of the blocks may be surrounded by the other block, as shown in FIG. 3.

Each of the blocks A and B may present a color difference ΔE between color within the stick and color after application on keratinous materials.

Only one of the blocks A and B need present this characteristic, it being possible for the other block not to present this property.

The two blocks A and B may optionally be of substantially the same color within the stick, e.g. white in color.

Where appropriate, the stick may have more than two blocks, for example three blocks as shown in FIG. 4. The third block E may optionally present a color difference between color within the stick, in its bulk, and color after application on keratinous materials.

The stick may be made by coextrusion, casting, or compacting compositions that are to form the various blocks.

In the example of FIG. 5, the cosmetic comprises two blocks A and B that have been cast or compacted in a cup.

In a variant that is not shown, the blocks of composition are contained in a sheath so as to form a crayon.

Each block A or B may be of cross-section that is constant along the entire width of the cosmetic. In a variant, one of the blocks at least may present a cross-section that varies along the length of the cosmetic.

One of the blocks may present a cross-section that is circular.

In longitudinal section, one of the blocks may present a chamfered shape flaring towards one longitudinal end while the other block also presents a chamfered shape, flaring in the opposite direction, as shown by the longitudinal section of FIG. 6.

The makeup may be applied without mixing the compositions on application, for example.

By way of example, the stick may be moved in translation so as to leave two colored traces having different colors and each corresponding to depositing the composition of one of the blocks.

Application may also be performed in such a manner as to cause the compositions of the blocks or superpose during application.

In a variant in which the blocks of composition are contained in a cup, each of the compositions may be taken by means of an applicator or a finger, for example.

### Example Proposed

The contents are given as percentages by weight.

### Example 24

### Lipstick

First composition

| | |
|---|---|
| Tridecyl trimellitate | 11 |
| Liquid lanolin | 10 |
| Isostearyl malate | 12 |
| Acetylated lanolin | 10 |
| Triglycerides of | 5 |
| lauric/palmitic/cetylic/stearic acids | |
| (50/20/10/10) | |
| (C₂₀-C₆₀) microcrystalline wax | 3 |
| Protected isopropyl lanolate | 9 |
| 2-Octyl decanol | 15 |
| Phenyl trimethylsiloxy trisiloxane (viscosity: | 4 |
| 20 cSt-MW: 372) | |
| Polyethylene wax (MW: 500) | 8 |
| Multilayer interference pigment* | 10 |
| Metallic reflection pigment** | 3 |

*TIMIRON SILK RED from MERCK.
**METASHINE ME 2040 PS from NIPPON SHEET GLASS

Second composition

| | |
|---|---|
| Tridecyl trimellitate | 11 |
| Liquid lanolin | 10 |
| Isostearyl malate | 12 |
| Acetylated lanolin | 10 |
| Triglycerides of lauric/palmitic/cetylic/stearic | 5 |
| acids (50/20/10/10) | |
| (C₂₀-C₆₀) microcrystalline wax | 3 |
| Protected isopropyl lanolate | 9 |
| 2-Octyl decanol | 15 |
| Phenyl trimethylsiloxy trisiloxane (viscosity: | 4 |
| 20 cSt-MW: 372) | |
| Polyethylene wax (MW: 500) | 8 |
| Multilayer interference pigment* | 10 |
| Metallic reflection pigment** | 3 |

*TIMIRON SILK BLUE from MERCK.
**METASHINE ME 2040 PS from NIPPON SHEET GLASS

The two compositions are coextruded to form two blocks that produce different colors on application.

The red, first composition occupies the center of the stick, for example, while the blue, second composition occupies its periphery, with the two blocks being concentric. Violet can appear on application in the center, between blue borders.

Naturally, the invention is not limited to the examples given above. The term “comprising a” is synonymous with the term “comprising at least one”, and “lying in the range” should be understood as including the limits of the range.

Although the present invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed is:

1. A cosmetic composition including, in a cosmetically-acceptable medium, at least one multilayer interference pigment and at least one of:
a coloring agent that is sensitive to at least one external stimulus,
a reflecting pigment with metallic reflection and
a diffracting pigment,
the coverage of the composition being greater than or equal to 25, better 30, the multilayer interference pigment being at a content that imparts to the composition, a difference in color ΔE between the bulk color and the color after application that is greater than or equal to 5.

2. A composition according to claim 1, comprising a coloring agent that is sensitive to at least one external stimulus.

3. A composition according to claim 1, comprising a reflecting pigment with metallic reflection.

4. A composition according to claim 1, comprising a diffracting pigment.

5. A composition according to claim 1, the coloring agent that is sensitive to at least one external stimulus being suitable in one state for taking on a bulk color of dominant wavelength that differs from the dominant wavelength of the composition by an amount Δλ of less than 30 nm.

6. A composition according to claim 1, the coloring agent that is sensitive to at least one external stimulus being suitable in one state for taking on a color of dominant wavelength that differs from the dominant wavelength of the composition, after application to keratinous materials, by an amount Δλ of less than 30 nm.

7. A composition according to claim 1, the coloring agent that is sensitive to at least one external stimulus being colorless in one state.

8. A composition according to claim 7, the state corresponding to the presence of the stimulus.

9. A composition according to claim 7, the state corresponding to the absence of the stimulus.

10. A composition according to claim 8, the state existing within the bulk of the composition before application to keratinous materials.

11. A composition according to claim 8, the state existing after application to keratinous materials.

12. A composition according to claim 1, the coloring agent that is sensitive to at least one external stimulus being in solution in the medium.

13. A composition according to claim 1, the coloring agent that is sensitive to at least one external stimulus being dispersed in particulate form in the medium.

14. A composition according to claim 1, the coloring agent that is sensitive to at least one external stimulus being a photochromic agent.

15. A composition according to claim 1, the coloring agent that is sensitive to at least one external stimulus being a thermochromic agent.

16. A composition according to claim 1, the coloring agent that is sensitive to at least one external stimulus being a solvatochromic agent.

17. A composition according to claim 1, the coloring agent that is sensitive to at least one external stimulus being a piezochromic, tribochromic, or mechanoluminescent agent.

18. A composition according to claim 1, the ratio ml/m₂ between the content by weight ml of multilayer interference pigment and the total content by weight m₂ of coloring agent that is sensitive to an external stimulus or of reflecting pigment with metallic reflection or of diffracting pigment lying in the range 0.1 to 1.5.

19. A composition according to claim 1, the multilayer interference pigment including a substrate made of silica, mica, glass, alumina, or metal.

20. A composition according to claim 1, the composition being anhydrous.

121. A composition according to claim 1, the composition being aqueous.

22. A composition according to claim 1, the composition not being powder and the total percentage by weight of multilayer interference pigment lying in the range 7% to 20%, better in the range 8 to 15%.

23. A composition according to claim 22, the composition being liquid.

24. A composition according to claim 22, the composition being in stick form.

25. A composition according to claim 1, the composition being powder and the total percentage by weight of multilayer interference pigment lying in the range 40% to 95%.

26. A composition according to claim 1, the bulk color of the composition being white while the composition is not exposed to the stimulus.

27. A composition according to claim 26, the composition having, within its bulk, a whiteness index that is greater than or equal to 40.

28. A composition according to claim 1, the composition not including any coloring agent other than the multilayer interference pigment(s) and the coloring agent(s) that are sensitive to an external stimulus, or
the reflecting pigment(s) with metallic reflection, or
the diffracting pigment(s).

29. A composition according to claim 1, the multilayer interference pigment comprising at least four layers.

30. A composition according to claim 1, the multilayer interference pigment including a substrate made of a transparent material.

31. A composition according to claim 1, the multilayer interference pigment including a substrate made of silica, mica, or glass.

32. A composition according to claim 1, the color produced by the multilayer interference pigment being of dominant wavelength lying outside the range 580 nm to 650 nm.

33. A composition according to claim 1, including only a single multilayer interference pigment.

34. A composition according to claim 1, including at least two multilayer interference pigments.

35. A composition according to claim 34, both multilayer interference pigments having layers that are made of the same material, but with at least one layer of one pigment having a thickness that is different from a corresponding layer of the other pigment.

36. A cosmetic composition that is not a powder, said composition including, in a cosmetically-acceptable medium:
one or more multilayer interference pigments at a content by weight lying in the range 7% to 20% of the total weight; and
one or more coloring agents that is/are sensitive to at least one external stimulus or one or more reflecting pigments with metallic reflection or one or more diffracting pigments, at a content by weight lying in the range 0.1% to 10%.

37. A composition according to claim 36, the composition being in stick form.

38. A composition according to claim 36, the composition being liquid.

39. A composition according to claim 36, the total percentage by weight of multilayer interference pigment(s) lying in the range 8% to 15%.

40. A cosmetic composition that is a powder, said composition including, in a cosmetically-acceptable medium:
one or more multilayer interference pigments at a content by weight lying in the range 40% to 95% of the total weight; and
one or more coloring agents that is/are sensitive to at least one external stimulus or one or more reflecting pigments with metallic reflection or one or more diffracting pigments, at a content by weight lying in the range 0.1% to 60% of the total weight.

41. A composition according to claim 40, the composition including multilayer interference pigment(s) at a content by weight lying in the range 55% to 65%.

42. A composition according to claim 40, the composition including a coloring agent(s) that is/are sensitive to at least one external stimulus or a reflecting pigment with metallic reflection or a diffracting pigment, at a content by weight lying in the range 1% to 50% of the total weight.

43. A composition according to claim 1 comprising less than 0.5% by weight of pigments generating a color by a phenomenon of absorbing light.

44. A composition according to claim 43, the composition comprising less than 0.5% by weight of pigments comprising iron oxide.

45. A composition according to claim 1, the multilayer interference pigment being deprived of a colored layer.

46. A composition according to claim 1, the multilayer pigment only comprising on the substrate one or several layers chosen from TiO2, ZrO2, SnO2, SiO2.

47. A composition according to claim 1, the multilayer interference pigment being deprived of Fe2O3.

48. A composition according to claim 1, the weight proportion of the multilayer interference pigment being greater than 7%.

49. A composition according to claim 3, the reflecting pigment with metallic reflection including a surface layer of metal or metal alloy.

50. A composition according to claim 3, the reflecting pigment with metallic reflection including a surface layer of metal oxide.

51. A composition according to claim 3, the reflecting pigment with metallic reflection being in the form of flakes.

52. A composition according to claim 3, the reflecting pigment with metallic reflection being in substantially spherical form.

53. A composition according to claim 3, the size of the reflecting pigment with metallic reflection lying in the range 10 μm to 500 μm.

54. A composition according to claim 3, the size of the reflecting pigment with metallic reflection lying in the range 10 μm to 150 μm better in the range 40 μm to 150 μm.

55. A composition according to claim 3, the ratio m₁/m₂ of the percentage by weight m₁ of multilayer interference pigment over the percentage by weight m₂ of the reflecting pigment with metallic reflection lying in the range 0.1 to 1.5.

56. A composition according to claim 3, the reflecting pigment with metallic reflection presenting a multilayer structure.

57. A composition according to claim 3, the multilayer interference pigment and the reflecting pigment with metallic reflection presenting substrates of the same material.

58. A composition according to claim 4, the percentage by weight of diffracting pigment lying in the range 0.1 to 60.

59. A composition according to claim 4, the diffracting pigment presenting a multilayer structure.

60. A composition according to claim 4, the multilayer interference pigment and the diffracting pigment presenting a substrate of the same material.

61. Packaging comprising:
a composition as defined in claim 1; and
means for informing the user of the color of the composition after application to keratinous materials.

62. A method of making up keratinous materials, the method comprising applying, to keratinous materials, a composition according to claim 1.
